(19) United States
(12) Patent Application Publication
(10) Pub. No.: US 2023/0302159 A1
(43) Pub. Date: Sep. 28, 2023

(54) COMPOUNDS AND COMPOSITIONS FOR TUMOR DETECTION AND SURGICAL GUIDANCE

(71) Applicants: Cornell University, Ithaca, NY (US); Molecular Targeting Technologies, Inc., West Chester, PA (US)

(72) Inventors: Ching-Hsuan Tung, New York, NY (US); Brian D. Gray, Exton, PA (US); Koon Yan Pak, Malvern, PA (US)

(21) Appl. No.: 18/041,532

(22) PCT Filed: Aug. 16, 2021
(86) PCT No.: PCT/US21/46181

§ 371 Date: Feb. 13, 2023

Related U.S. Application Data
(60) Provisional application No. 63/066,072, filed on Aug. 14, 2020.

(51) Int. Cl. A61K 49/00; C09K 11/06; C09B 23/01

(52) U.S. Cl. CPC A61K 49/0032; C09K 11/06; A61K 49/006; C09B 23/0066; C09K 2211/1018

(57) ABSTRACT

Disclosed are compounds with the following structure (Formula (I)) where (Formula (II)) or (Formula (III)), X is an anion (e.g., a biologically suitable anion, such as, for example, chloride, iodide, and the like). Y is NH, NR¹⁰, or CR¹¹R¹². Z is a heteroatom (e.g., O, S, or Se). R and R¹ are independently chosen from methyl, ethyl, propyl (e.g., n-propyl, isopropyl), butyl (e.g., n-butyl, isobutyl, tert-butyl), and the like, and combinations thereof. In various examples, R and R¹ are not both oxygen atoms (such that an —NO₂ is formed). R² and R³ are independently chosen from methyl, ethyl, propyl (e.g., n-propyl, isopropyl), butyl (e.g., n-butyl, isobutyl, tert-butyl), and the like, and the like, and combinations thereof. R⁴, R⁵, R⁶, R⁷, R⁸, R⁹, R¹⁰, R¹¹, and R¹² are independently chosen from hydrogen, alkyl groups (e.g., methyl, ethyl, propyl (e.g., n-propyl, isopropyl), butyl (e.g., n-butyl, isobutyl, tert-butyl)), and the like, and combinations thereof. Also disclosed are compositions and methods of using the compounds and compositions. The compounds or compositions may be used to visualize or identify tumors.

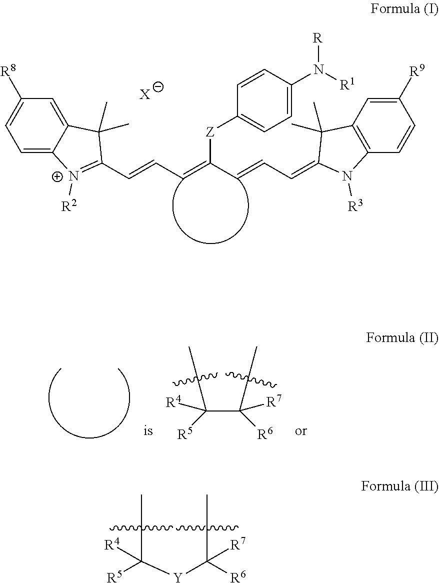

### CROSS REFERENCE TO RELATED APPLICATION

This application claims priority to U.S. Provisional Application No. 63/066,072, filed on Aug. 14, 2020, the disclosure of which is incorporated herein by reference.

### BACKGROUND OF THE DISCLOSURE

Surgical resection of cancerous tissues is a critical procedure for solid tumor treatment. Surgery is one of the most effective ways of treating solid tumors. If all cancerous tissues could be removed during the procedure, the chance of having an extended disease-free period or even a cure is high. However, some cancerous tissues are not apparent to naked-eye surveillance, hence the surgical outcome could vary.

Even though preoperative imaging by MRI, CT, and PET have significantly improved the efficacy of cancer diagnosis and treatment planning, current intraoperative surgery still relies heavily on the surgeon's experience, skill, thoroughness, and patience. Surgeons mostly use visual inspection and palpation to identify possible cancerous tissues; however, these two methods are often inadequate. During the operation, the surgeon mostly identifies the cancerous tissues by naked-eye visualization under white light without aid, therefore, the outcome heavily relies on the surgeon's experience. Large tumors which are readily seen by naked eye surveillance can be promptly removed, but the smaller lesions which may be imperceptible could be left behind. Incomplete resection can lead to disease recurrence and over resection may cause surgical complications, therefore fluorescence-guided surgery (FGS), an emerging technology, is actively being tested to augment accuracy, efficacy, and efficiency of intraoperative operations. FGS require a fluorescent probe to enhance the visibility of cancerous tissues and a fluorescence imaging system for real-time detection of the fluorescence signal. Although, non-targeted conventional fluorescent dyes such as indocyanine green, methylene blue, and fluorescein have been FDA-approved to track blood, lymph, and urine flows, they have limited utility in cancer imaging due to the passive nature of their accumulation in tumors. By taking advantage of a cancer's unique physiological characteristics (e.g., high receptor expression and high enzyme activity), various fluorescent probes are designed to highlight the cancerous tissues. Fluorophores have been conjugated to targeting ligands or responsive triggers to construct tumor-binding or enzyme-activatable fluorescent probes. Systemically administered fluorescent probes specifically illuminate the cancerous tissues, not the normal tissues, by either preferential binding or enzymatic activation, resulting in a high tumor to normal tissue contrast. Several promising fluorescent probes currently are undergoing FGS clinical trials.

For maximal signal to background contrast, most probes are intravenously (IV) injected several hours or day(s) before imaging to allow the background signal clearance of unbound probes or the signal generation of enzyme activatable probes. Patients, therefore, must be admitted to hospital for contrast agent injection many hours or even days before the actual surgical procedure which extends their time in hospital. Furthermore, IV administered probes may have limited sensitivity for small tumors (<2 mm) because they may not reach them due to the tumor's underdeveloped vasculature. Since small tumors are already easily overlooked by naked-eye surveillance during the procedure and could be a source of recurrence, systemic agents might not help the situation. Systemic administration of probe also requires a large dosage which may cause systemic side-effects.

Typical “always-on” fluorescence binding probes that have a fast on-rate to tumor do not fit well with the “spray-and-see” approach because any extra agent applied on normal tissue has to be washed away before imaging. Conversely, low background enzyme activatable probes avoid the washing step but the slow catalytic enzyme reaction prohibits the immediate imaging possibility. Tumor cells usually exhibit enhanced glycolysis to maintain their rapid growth and proliferation, and the aerobic environment in solid tumors alters their metabolic pathway to convert glucose to lactic acid instead of pyruvate. They actively pump out protons to reduce the intracellular lactic acid build-up, which ultimately leads to a significant decrease of extracellular pH in tumors from 7.4 to 6.2-6.9. Tumor acidity is correlated to enhance tumor growth, invasiveness, and metastasis. This tumor-associated acidity has also been used to develop a number of IV delivered pH-responsive fluorescent probes by conjugating a pH-sensitive dye to a tumor-targeting group such as an antibody or peptide. Their tumor-specific binding and internalization into the acidic endosomes and lysosomes (pH=4.5-5.0) make the tumor emit strong fluorescence. However, probes of this type suffer from similar drawbacks to the common IV administered probes.

### BRIEF SUMMARY OF THE DISCLOSURE

In an aspect, the present disclosure provides compounds. The compounds may be used to visualize (e.g., highlight) cancerous tissues during a procedure.

In various examples, the present disclosure provides compounds having the following structure:

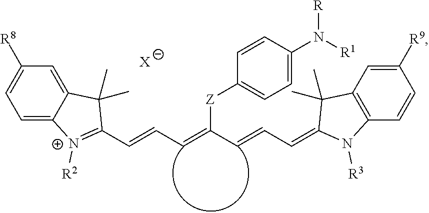

where

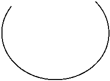

is

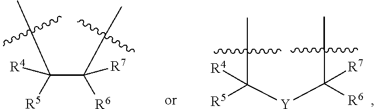

X is an anion (e.g., a biologically suitable anion, such as, for example, chloride, iodide, and the like). Y is NH, NR¹⁰, or CR¹¹R¹². Z is a heteroatom (e.g., O, S, or Se). R and R¹ are independently chosen from methyl, ethyl, propyl (e.g., n-propyl, isopropyl) butyl (e.g., n-butyl, isobutyl, tert-butyl), and the like, and combinations thereof. In various examples, R and R¹ are not both oxygen atoms (such that an —NO₂ is formed). In various examples, R and R¹ are not both hydrogen atoms. R² and R³ are independently chosen from methyl, ethyl, propyl (e.g., n-propyl, isopropyl), butyl (e.g., n-butyl, isobutyl, tert-butyl), and the like, and combinations thereof. R⁴, R⁵, R⁶, R⁷, R⁸, R⁹, R¹⁰, R¹¹, and R¹² are independently chosen from hydrogen, alkyl groups (e.g., methyl, ethyl, propyl (e.g., n-propyl, isopropyl), butyl (e.g., n-butyl, isobutyl, tert-butyl)), and the like, and combinations thereof. In various examples, R⁴ and R⁵ may be the same alkyl group (e.g., methyl groups). In various examples, R⁶ and R⁷ may be the same alkyl group (e.g., methyl groups).

In an aspect, the present disclosure provides compositions comprising one or more compounds of the present disclosure. The compositions may comprise one or more pharmaceutically acceptable carriers.

In an aspect, the present disclosure provides methods of using one or more compounds or compositions of the present disclosure. Methods of the present disclosure may be used on an individual having or suspected of having cancer (e.g., a solid tumor). The methods may be used to detect, identify, visualize, or otherwise image a solid tumor.

### BRIEF DESCRIPTION OF THE FIGURES

For a fuller understanding of the nature and objects of the disclosure, reference should be made to the following detailed description taken in conjunction with the accompanying figures herein.

FIG. 1 shows compounds of the present disclosure.

FIG. 2 shows compounds of the present disclosure.

FIG. 3 shows emission maximum and intensity difference at pH 5.0 and 7.5.

FIG. 4 shows fluorescence spectra of compounds of the present disclosure.

FIG. 5 shows cytotoxicity data of the compounds of the present disclosure. A CCK8 MMT assay was performed using 1 μM of each compound with 0.1% DMSO with RPMI. Cells were incubated for 0.5 or 1 hour, washed with fresh media, and then incubated for 3 days.

FIG. 6 shows absorbance and fluorescence spectra of CypH-11 (2 μM in pH 5.0 phosphate buffer solution. Exₘₐₓ=766 nm and Emₘₐₓ=785 nm.

FIG. 7 shows a comparison of pH responsive CypH-11 and pH insensitive Cy7.

FIG. 8 shows a comparison of CypH-1 and CypH-11.

FIG. 9 shows tumor/muscle contrast ratio of CypH-11, CypH-1 and Cy7 at different time points.

FIG. 10 shows a general synthesis scheme for compounds of the present disclosure.

FIG. 11 shows a synthesis scheme for CypH-11.

FIG. 12 shows chemical structures and characterization of CypH-11 and CypH-1. (A) The structure of CypH-1 (B) Schematic diagram of the fluorescence activation of CypH-11 by protonation. (C) Measurement of pKa values of CypH-11 and CypH-1 (6.0 and 4.7 respectively). Their fluorescence intensity in the pH range (2.0-11.0) was measured (λₑₓ=725 nm and λₑₘ=785 nm) using a plate reader. (D) Fluorescence image of CypH-11 and CypH-1 in a 96-well plate at different pHs.

FIG. 13 shows cellular imaging and intracellular localization of CypH-11, CypH-1, and Cy7. (A) OVASAHO cells were incubated with CypH-11, CypH-1, and Cy7 (2 μM each) for 1 hr and cell images were captured without washing. Scale bar: 50 μm. (B) Colocalization of CypH-11 and CypH-1 with mitochondria and lysosome. OVASAHO cells were incubated with CypH-11 and CypH-1 (2 μM each) for 1 hr, then cells were further stained with an organelle tracker (mitochondria or lysosome tracker) for 10 min. Cell images were captured with a NIR channel (excitation: 690-730 nm and emission: 770-850 nm) and GFP channel (excitation: 457-487 nm and emission: 502-538 nm). Scale bar: 50 μm. (C) Cell viability of CypH-11 and CypH-1. OVASAHO cells were firstly treated with CypH-11 or CypH-1 (2 μM each) for 1 hr, and the cells were grown for an additional 72 hrs after replacing the old medium with a fresh cell culture medium. Cell viability was evaluated with the CCK assay. Each column represents the average of three separate experiments.

FIG. 14 shows in vivo and ex vivo images of CypH-11, CypH-1, and Cy7 in the subcutaneous OVASAHO/RFP-Luc tumor model. (A) Representative white light and fluorescence composite images of nude mice at various time points before and after spraying of CypH-11 and Cy7 (2 μM each) on the surgical area (10 tumors for CypH-11 and 3 tumors for Cy7). RFP images indicated the tumor size and location, and the NIR images showed the CypH-11 and Cy7 fluorescence changes after the spraying. Scale bar=1 cm. (B) White light and fluorescence composite images before and after spraying of CypH-11 (2 μM, right flank) and CypH-1 (2 μM, left flank). Scale bar=1 cm. (C) Ex vivo fluorescence images of the excised tumors and muscles after spraying the probe. Scale bar=1 cm. (D) Tumor-to-normal tissue ratio of fluorescence at different time points after spraying the probes on the surgical area.

FIG. 15 shows in vivo images of CypH-11 in the subcutaneous SKOV3/GFP-Luc tumor model. (A) Representative white light and fluorescence composite images of a nude mice at various time points before and after spraying of CypH-11 (2 μM, 6 tumors). The GFP image indicated the tumor location and size, and the CypH-11 image indicated the NIR fluorescence produced by CypH-11. Scale bar=1 cm. (B) Tumor-to-normal tissue ratio of fluorescence intensity at different time points after spraying CypH-11 on the surgical area. N=6. (C) Histological correlation of the fluorescent signal of tumor and CypH-11 signal. GFP and DAPI signals were across the whole tumor section, and NIR signal from CypH-11 was only on the edge. Scale bar=100 μm.

FIG. 16 shows in vivo and ex vivo white light and fluorescence composite images of the disseminated SKOV3/RFP-Luc tumors in the peritoneal cavity after IP administration of CypH-11. (A) Three representative mice were imaged 1 h post IP administration of CypH-11 (200 μL 2 μM solution). GFP images indicated the location and size of the disseminated tumors (top row), and CypH-11 images indicated the fluorescence signal produced by CypH-11 (bottom row). Scale bar=1 cm. (B) Ex vivo white light and fluorescence composite images of the excised tumor-bearing organs (tumor, spleen, stomach, liver, and intestine). Scale bar=5 mm. (C) Tissue-to-peritoneum ratio of fluorescence intensity of SKOV3 mice post IP administration. (D) Histological correlation of the fluorescent signal of tumor and CypH-11 signal. Scale bar=100 μm.

FIG. 17 shows chemical synthesis and spectra of CypH-11. (A) Synthetic scheme of CypH-11 (B) Normalized absorption and emission spectra of CypH-11 at pH=5 PBS buffer, EXₘₐₓ=765 nm; Emₘₐₓ=785 nm.

FIG. 18 shows the chemical structure and optical property of Cy7 from GE Healthcare. (A) Structure of Cy7. (B) Measurement of fluorescence intensity of Cy7 at different pHs (λₑₓ=725 nm and λₑₘ=785 nm) using a plate reader. (D) Fluorescence image of Cy7 in a 96-well plate at different pHs.

FIG. 19 shows OVASAHO cells incubated with CypH-11, CypH-1, and Cy7 (2 μM each) for 1 hr, washed with PBS and then imaged. Scale bar: 50 μm. Cell images were captured with a NIR channel (excitation: 690-730 nm and emission: 770-850 nm).

FIG. 20 shows depth determination of CypH-11 signal in sprayed and IP injected tumors (40×). (A) The nucleus DAPI stain showed the sprayed CypH-11 can only penetrate 2-3 layers of cells in 15 min. (B) The IP injected CypH-11 was able to reach 6-7 layers of cells in an hour. Scale bar =50 μm.

FIG. 21 shows CypH-11 signal development in live and dead tissues. (A) In vivo spray. CypH-11 was sprayed onto the tissues in live animals first and then the tissues were excised 20 min later. A good correlation of the tumor GFP and NIR signals was observed. (B) Ex vivo spray. The tissues were excised, held for 20 min and then sprayed with CypH-11. No CypH-11 signal in tumor or muscle was observed, indicating that dead tissues cannot generate a CypH-11 signal, probably due to poor uptake of the probe. Scale bar=5 mm.

FIG. 22 show characterization data of CypH-11. (A) ¹ NMR; (B) ¹³C NMR; and (C) mass analysis.

### DETAILED DESCRIPTION OF THE DISCLOSURE

Although claimed subject matter will be described in terms of certain examples, other examples, including examples that do not provide all of the benefits and features set forth herein, are also within the scope of this disclosure. Various structural, logical, and process step changes may be made without departing from the scope of the disclosure.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value) of a range.

As used herein, unless otherwise stated, the term “group” refers to a chemical entity that is monovalent (i.e., has one terminus that can be covalently bonded to other chemical species), divalent, or polyvalent (i.e., has two or more termini that can be covalently bonded to other chemical species). The term “group” also includes radicals (e.g., monovalent and multivalent, such as, for example, divalent radicals, trivalent radicals, and the like). Illustrative examples of groups include:

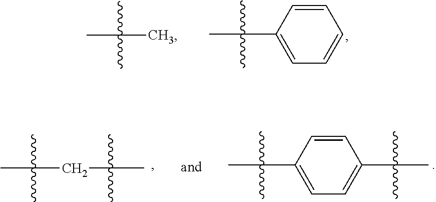

As used herein, unless otherwise indicated, the term “alkyl group” refers to branched or unbranched saturated hydrocarbon groups. Examples of alkyl groups include, but are not limited to, methyl groups, ethyl groups, propyl groups, butyl groups, isopropyl groups, tert-butyl groups, and the like. For example, the alkyl group is C₁ to C₂₀, including all integer numbers of carbons and ranges of numbers of carbons therebetween (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, and C₂₀). The alkyl group may be unsubstituted or substituted with one or more substituents. Examples of substituents include, but are not limited to, various substituents such as, for example, halogens (—F, —Cl, —Br, and —I), aliphatic groups (e.g., alkyl groups, alkenyl groups, alkynyl groups, and the like), aryl groups, alkoxide groups, carboxylate groups, carboxylic acids, ether groups, amine groups, and the like, and combinations thereof.

The present disclosure provides compounds and compositions suitable to visualize solid tumors. A compound of the present disclosure or composition comprising a compound may be used to visualize (e.g., highlight) cancerous tissues during a procedure (e.g., medical procedure, such as, for example, surgery (e.g., tumor removal)). Visualization may be used to minimize undesired overlook and overall achieve better surgical outcome. Also provided are methods of using the compounds and compositions.

In an aspect, the present disclosure provides compounds. The compounds may be used to visualize (e.g., highlight) cancerous tissues during a procedure.

In various examples, the present disclosure provides compounds having the following structure:

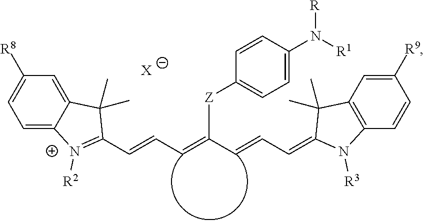

where

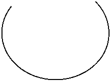

is

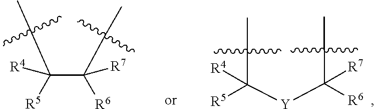

X is an anion (e.g., a biologically suitable anion, such as, for example, chloride, iodide, and the like). Y is NH, NR¹⁰, or CR¹¹R¹². Z is a heteroatom (e.g., O, S, or Se). R and R¹ are independently chosen from methyl, ethyl, propyl (e.g., n-propyl, isopropyl) butyl (e.g., n-butyl, isobutyl, tert-butyl), and the like, and combinations thereof. In various examples, R and R¹ are not both oxygen atoms (such that an —NO₂ is formed). In various examples, R and R¹ are not both hydrogen atoms. R² and R³ are independently chosen from methyl, ethyl, propyl (e.g., n-propyl, isopropyl), butyl (e.g., n-butyl, isobutyl, tert-butyl), and the like, and the like, and combinations thereof. R⁴, R⁵, R⁶, R⁷, R⁸, R⁹, R¹⁰, R¹¹, and R¹² are independently chosen from hydrogen, alkyl groups (e.g., methyl, ethyl, propyl (e.g., n-propyl, isopropyl), butyl (e.g., n-butyl, isobutyl, tert-butyl)), and the like, and combinations thereof. In various examples, R⁴ and R⁵ may be the same alkyl group (e.g., methyl groups). In various examples, R⁶ and R⁷ may be the same alkyl group (e.g., methyl groups).

In various examples, the present disclosure provides compounds having the following structure:

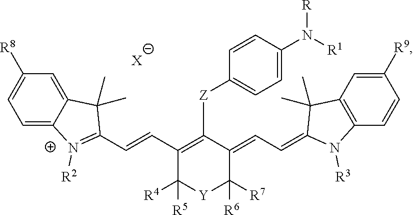

where X is an anion (e.g., a biologically suitable anion, such as, for example, chloride, iodide, and the like). Y is NH, NR¹⁰, or CR¹¹R¹². Z is a heteroatom (e.g., O, S, or Se). R and R¹ are independently chosen from methyl, ethyl, propyl (e.g., n-propyl, isopropyl), butyl (e.g., n-butyl, isobutyl, tert-butyl), and the like, and combinations thereof. In various examples, R and R¹ are not both oxygen atoms (such that an —NO₂ is formed). In various examples, R and R¹ are not both hydrogen atoms. R² and R³ are independently chosen from methyl, ethyl, propyl (e.g., n-propyl, isopropyl), butyl (e.g., n-butyl, isobutyl, tert-butyl), and the like, and combinations thereof. R⁴, R⁵, R⁶, R⁷, R⁸, R⁹, R¹⁰, R¹¹, and R¹² are independently chosen from hydrogen, alkyl groups (e.g., methyl, ethyl, propyl (e.g., n-propyl, isopropyl), butyl (e.g., n-butyl, isobutyl, tert-butyl)), and the like, and combinations thereof. A compound of the present disclosure does not have the following structure:

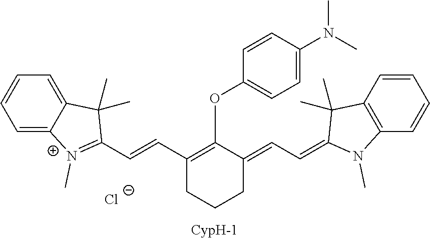 [SMILES: CN(C)c1ccc(OC2=C(/C=C/C3=[N+](C)c4ccccc4C3(C)C)CCC/C2=C\C=C2\N(C)c3ccccc3C2(C)C)cc1 ; [Cl-]]

Without intending to be bound by any particular theory. The compounds of the present disclosure are pH sensitive. The compounds may be non-fluorescent in normal tissues, but fluoresce when absorbed by cancer tissue, which is acidic. The cancer preferential staining capability will make the surgical procedure precise and effective. Medical professionals could locate tumors accurately regardless of their size and shape, and execute all necessary procedures with precision in a timely manner.

The compounds of the present disclosure have a desirable pKa value. A compound may have a pKa in the range of 5.5-6.5, including all values and ranges therebetween. Compounds with pKa values below 5 may not possess the desirable fluorescence for topical application (e.g., spray application).

Examples of compounds of the present disclosure include, but are not limited to:

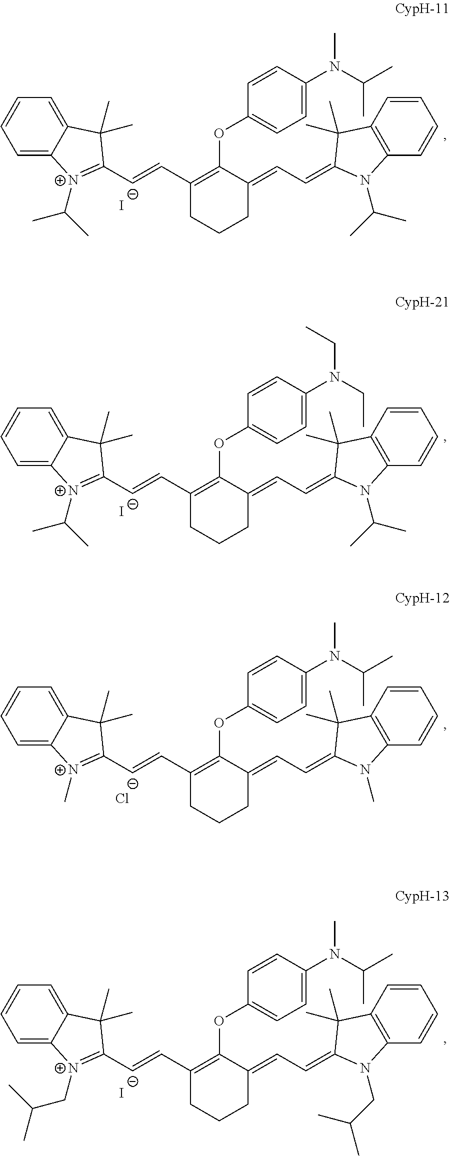

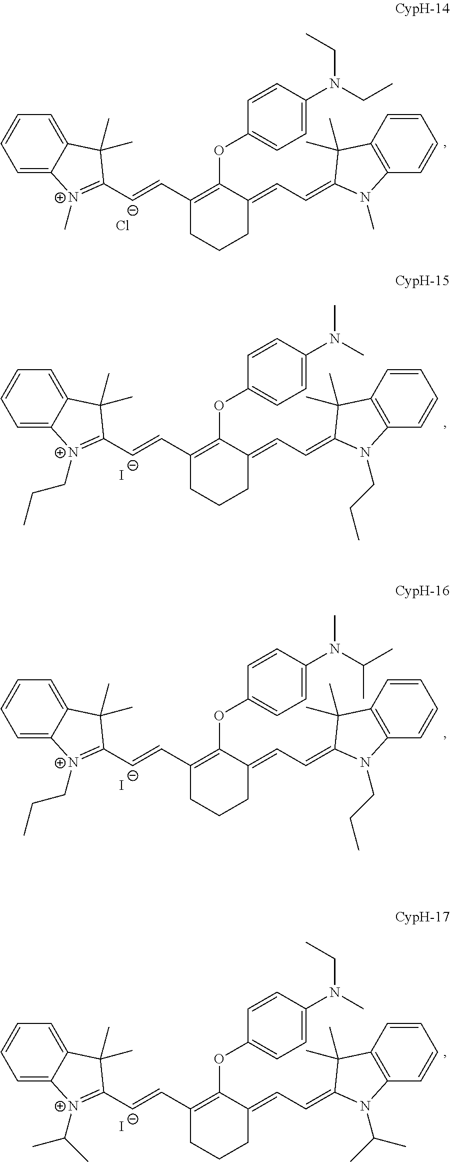

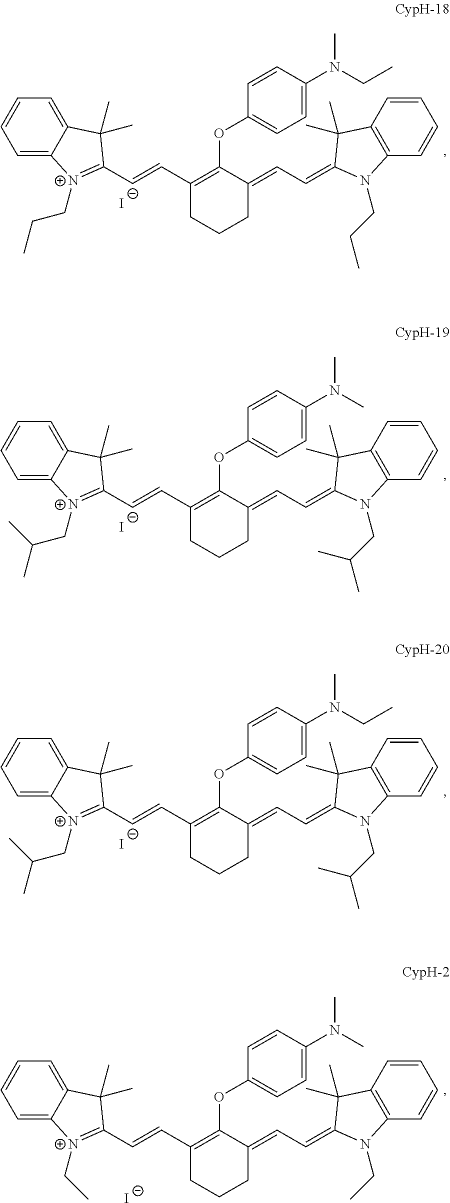

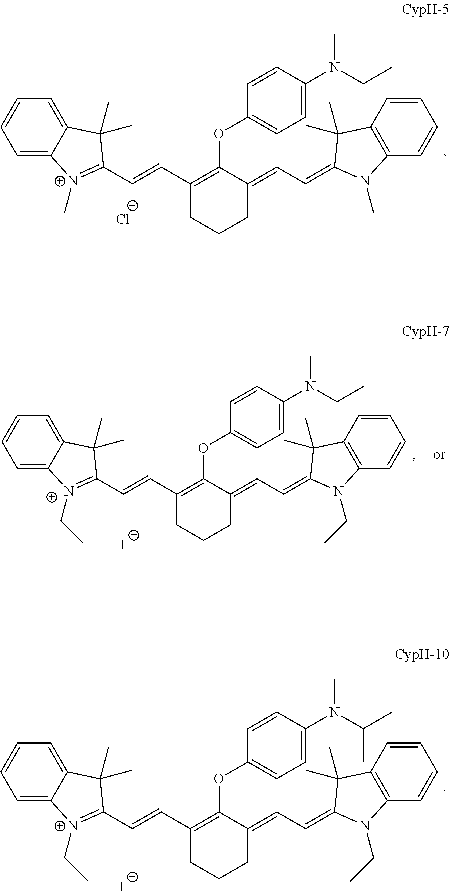

In an aspect, the present disclosure provides compositions comprising one or more compounds of the present disclosure. The compositions may comprise one or more pharmaceutically acceptable carriers.

The compositions described herein may include one or more standard pharmaceutically acceptable carriers. Pharmaceutically acceptable carriers may be determined in part by the particular composition being administered, as well as by the particular method used to administer the composition. Accordingly, there are a wide variety of suitable formulations of pharmaceutical compositions of the present disclosure. The compounds may be freely suspended in a pharmaceutically acceptable carrier or the compounds may be encapsulated in liposomes and then suspended in a pharmaceutically acceptable carrier. Examples of carriers include solutions, suspensions, emulsions, solid injectable compositions that are dissolved or suspended in a solvent before use, and the like. The injections may be prepared by dissolving, suspending or emulsifying one or more of the active ingredients in a diluent. Examples of diluents, include, but are not limited to distilled water for injection, physiological saline, vegetable oil, alcohol, dimethyl sulfoxide, and a combination thereof. Further, the injections may contain stabilizers, solubilizers, suspending agents, emulsifiers, soothing agents, buffers, preservatives, etc. The injections may be sterilized in the final formulation step or prepared by sterile procedure. The composition of the disclosure may also be formulated into a sterile solid preparation, for example, by freeze-drying, and can be used after sterilized or dissolved in sterile injectable water or other sterile diluent(s) immediately before use. Additional examples of pharmaceutically acceptable carriers include, but are not limited to, sugars, such as lactose, glucose, and sucrose; starches, such as corn starch and potato starch; cellulose, including sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Additional non-limiting examples of pharmaceutically acceptable carriers can be found in: Remington: The Science and Practice of Pharmacy (2005) 21st Edition, Philadelphia, PA. Lippincott Williams & Wilkins. Effective formulations include, but are not limited to, oral and nasal formulations, topical formulations, formulations for parenteral administration, and compositions formulated for extended release. Parenteral administration includes infusions such as, for example, intramuscular, intravenous, intraarterial, intraperitoneal, subcutaneous administration, and the like.

In various examples, the composition has desirable permeation characteristics and biologically suitable osmolarity. Carriers with desirable permeation characteristics include, but are not limited to, propylene glycol, isopropanol, oleic acid, and polyethylene glycol analogs, and the like, and combinations thereof. It is desirable that the composition is non-lethal to cells. It is believed that osmolarity regulating agents may be used. Examples of osmolarity regulating agents include, but are not limited to, sugars (e.g., monosaccharides, such as, for example, glucose, fructose, sorbose, xylose, ribose, and the like, and combinations thereof, disaccharides, such as, for example, sucrose, sugar-alcohols, such as, for example, mannitol, glycerol, inositol, xylitol, adonitol, and the like, and combinations thereof, and amino acids, such as, for example, glycine, arginine, and the like and combinations thereof.

In various examples, the compositions are suitable for topical administrations. The compositions may be sprayed onto a subject having a solid tumor or suspected of having a solid tumor at location where it is believed the subject has a solid tumor or where the subject has a solid tumor or used as an oral rinse for oral and/or esophageal cancers. The spray could also be applied to assist endoscopic/laparoscopic diagnosis in patients with ovarian, colon, bladder, esophagus, cervical, oral and other cancers. The composition may be administered (e.g., sprayed) directly from an endoscope, colonoscope, or laparoscope. In various examples, a compound or composition may be administered in all surgical resection or to validate the excised tissues.

In various examples, the composition may comprise 0.5 to 10 μM of a compound of the present disclosure, including every 0.01 μM value and range therebetween, in phosphate buffered saline with a pH of 6.5 to 7.5, including every 0.01 pH value and range therebetween, and 0.1 to 1.0% by volume DMSO, including 0.01% by volume value and range therebetween. In various examples, the composition may comprise 0.5 to 10 μM compound, including every 0.01 μM value and range therebetween, in phosphate buffered saline with a pH of 6.5 to 7.5, including every 0.01 pH value and range therebetween.

In an aspect, the present disclosure provides methods of using one or more compounds or compositions of the present disclosure. Methods of the present disclosure may be used on an individual having or suspected of having cancer (e.g., a solid tumor). The methods may be used to detect, identify, visualize, or otherwise image a solid tumor.

Methods of the present disclosure may be used to determine the presence and/or location of a solid tumor and/or image a solid tumor. The methods may be used in combination with other methods used to identify or remove a solid tumor.

A method for determining the presence and/or location of a solid tumor in an individual may comprise administering a compound or a composition of the present disclosure to an area of interest on or in the individual. The area of interest may be an area where an individual has or is suspected of having a tumor. The compound or the composition is exposed (e.g., irradiated) with electromagnetic radiation (e.g., light have a wavelength in the near-infrared region (NIR) (e.g., 750 to 1500 nm)). Following irradiation, the area of interest may be imaged or visualized. Imaging or visualization may comprise measuring or observing a fluorescence signal at the area of interest. After application of the compound or composition, a signal may be detected within several minutes (e.g., less than 5 minutes, less than 4 minutes, less than 3 minutes, less than 2 minutes, less than 1 minute, less than 55 seconds, less than 50 seconds, less than 45 seconds, less than 40 seconds, less than 35 seconds, less than 30 seconds, less than 25 seconds, less than 20 seconds, less than 15 seconds, less than 10 seconds, or less than 5 seconds). In various examples, there is no washing prior to the imaging and/or visualizing. The fluorogenic signal will be developed in the neoplastic tumor tissue.

A method of the present disclosure may be a method of imaging a solid tumor. A method may comprise applying or administering a compound or composition of the present disclosure to a solid tumor, exposing the area of interest to electromagnetic radiation; and obtaining an image of the solid tumor. In various examples, there is no washing prior to the imaging and/or visualizing. After application of the compound or composition, a signal may be detected within several minutes (e.g., less than 5 minutes, less than 4 minutes, less than 3 minutes, less than 2 minutes, less than 1 minute, less than 55 seconds, less than 50 seconds, less than 45 seconds, less than 40 seconds, less than 35 seconds, less than 30 seconds, less than 25 seconds, less than 20 seconds, less than 15 seconds, less than 10 seconds, or less than 5 seconds). Administration may occur by various non-intravenous delivery methods, such as topical administration (e.g., sprayed on the area of interest) or intraperitoneal delivery (i.p.).

The presence, identification, and/or imaging of a tumor may comprise measuring a fluorescence signal. The excitation and emission may vary depending on the compound used to generate the fluorescence signal. The measuring may comprise measuring a background fluorescence. The signal may be measured at various time points (e.g., 1, 3, 5, 7, 10, and 15 minute time points). The measuring may be used to determine the tumor-to-normal tissue ratio by calculating the average fluorescence intensity of the tumor by that of the normal area.

Administration may occur by various non-intravenous delivery methods, such as topical administration (e.g., sprayed on the area of interest) or intraperitoneal delivery (i.p.). Further, the compounds or compositions of may be administered systemically. The term “systemic” as used herein includes parenteral, topical, oral, spray inhalation, rectal, nasal, and buccal administration. The term “parenteral” as used herein includes subcutaneous, intravenous, intramuscular, intra-articular, intra-synovial, intrasternal, intrathecal, intrahepatic, intralesional, and intracranial administration. In various examples, the compounds or compositions are applied or administered via topical application or topical administration. In various examples, the compounds or compositions are sprayed onto an area of interest. In various other examples, the compositions are an oral rinse. For example, the method may be a “spray and see” technique.

A method of the present disclosure may include determining the tumor margin of a tumor (e.g., solid tumor). For example, a compound or composition is applied to the site of a tumor (e.g., solid tumor) and measuring the fluorescence signal. The excitation and emission may vary depending on the compound used to generate the fluorescence signal. The measuring may comprise measuring a background fluorescence. The signal may be measured at various time points (e.g., 1, 3, 5, 7, 10, and 15 minute time points). The signal may be compared against the fluorescence signal of non-cancerous tissue in/of the area of interest to determine the margins of the tumor. The comparison may be used to determine which portions of the area of interest are cancerous and non-cancerous. The signal may be used to determine the tumor margin to ensure complete excision of the tumor.

Methods of the present disclosure may be used on various individuals. In various examples, an individual is a human or non-human mammal. Examples of non-human mammals include, but are not limited to, farm animals, such as, for example, cows, hogs, sheep, and the like, as well as pet or sport animals such as, for example, horses, dogs, cats, and the like. Additional non-limiting examples of individuals include, but are not limited to, rabbits, rats, mice, and the like. The compounds or compositions of the present disclosure may be administered to individuals for example, in pharmaceutically acceptable carriers, which facilitate transporting the compounds from one organ or portion of the body to another organ or portion of the body or may be applied directly to the organ or portion of the body of interest.

Various tumors may be identified, imaged, or visualized using a method of the present disclosure. For example, the tumors are solid tumors. Examples of tumors include, but are not limited to, ovarian tumors, skin cancer, pancreatic cancer, genitourinary cancer, colon tumors, bladder tumors, brain tumors, esophagus tumors, cervical tumors, oral tumors, and the like, and combinations thereof.

The steps of the methods described in the various embodiments and examples disclosed herein are sufficient to produce a compound of the present disclosure or carry out a method of the present disclosure. Thus, in various embodiments, a method consists essentially of a combination of the steps of the methods disclosed herein. In various other embodiments, a method consists of such steps.

In an aspect, the present disclosure provides kits. The kits may comprise a composition or the materials to prepare a composition (e.g., a pharmaceutical carrier and one or more compounds of the present disclosure) and printed material.

In various examples, a kit comprises a closed or sealed package that contains the pharmaceutical preparation. In various examples, the package comprises one or more closed or sealed vials, bottles, blister (bubble) packs, or any other suitable packaging for the sale, or distribution, or use of the compounds and compositions comprising compounds of the present disclosure. The printed material may include printed information. The printed information may be provided on a label, or on a paper insert, or printed on the packaging material itself. The printed information may include information that identifies the compound in the package, the amounts and types of other active and/or inactive ingredients, and instructions for taking the composition, such as the number of doses to take over a given period of time, and/or information directed to a pharmacist and/or another health care provider, such as a physician, or a patient. In various examples, the product includes a label describing the contents of the container and providing indications and/or instructions regarding use of the contents of the container. A kit may comprise a single dose or multiple doses. A kit may further comprise a device or article necessary to administer a compound or composition. The article or device may be, for example, a spray bottle or atomizer or the like.

The following examples are presented to illustrate the present disclosure. The examples are not intended to be limiting in any matter.

Example A. A compound having the following structure:

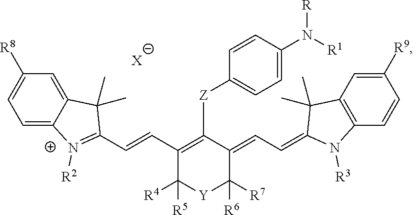

where X is an anion (e.g., a biologically suitable anion, such as, for example, chloride, iodide, or the like); Y is NH, NR¹⁰, or CR¹¹R¹²; Z is a heteroatom (e.g., O, S, or Se); R and R¹ are independently chosen from methyl, ethyl, propyl (e.g., n-propyl, isopropyl), butyl (e.g., n-butyl, isobutyl, tert-butyl), and the like, and combinations thereof; R² and R³ are independently chosen from methyl, ethyl, propyl (e.g., n-propyl, isopropyl), butyl (e.g., n-butyl, isobutyl, tert-butyl), and the like, and combinations thereof; R⁴, R⁵, R⁶, R⁷, R⁸, R⁹, R¹⁰, R¹¹, and R¹² are independently chosen from hydrogen, alkyl groups (e.g., methyl, ethyl, propyl (e.g., n-propyl, isopropyl), butyl (e.g., n-butyl, isobutyl, tert-butyl)), and the like, and combinations thereof, with the proviso the compound does not have the following structure:

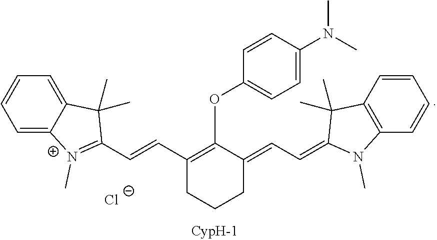 [SMILES: CN(C)c1ccc(OC2=C(/C=C/C3=[N+](C)c4ccccc4C3(C)C)CCC/C2=C\C=C2\N(C)c3ccccc3C2(C)C)cc1 ; [Cl-]]

E.g., in various examples, R⁴ and R⁵ may be the same alkyl group (e.g., methyl groups). E.g., in various examples, R⁶ and R⁷ may be the same alkyl group (e.g., methyl groups). In various examples, the compound has the following structure:

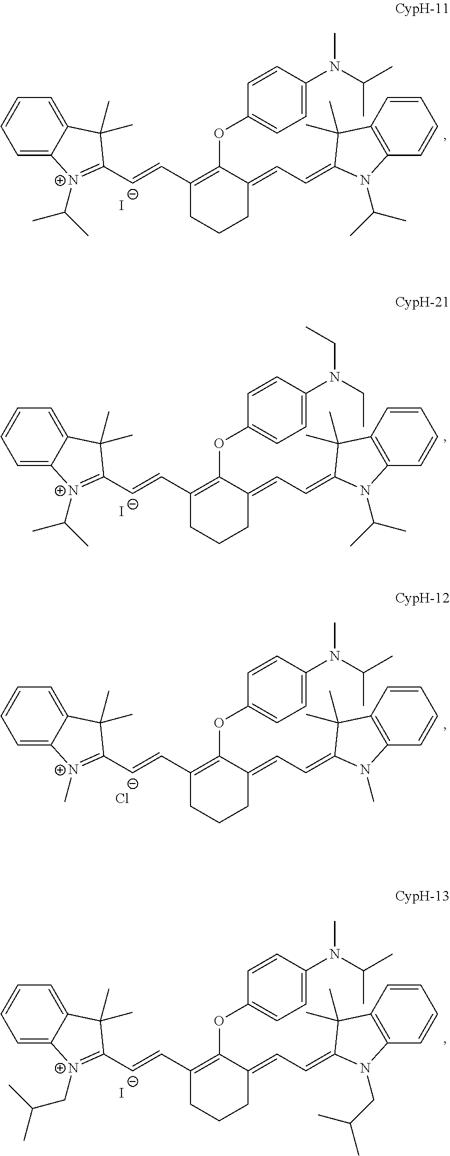

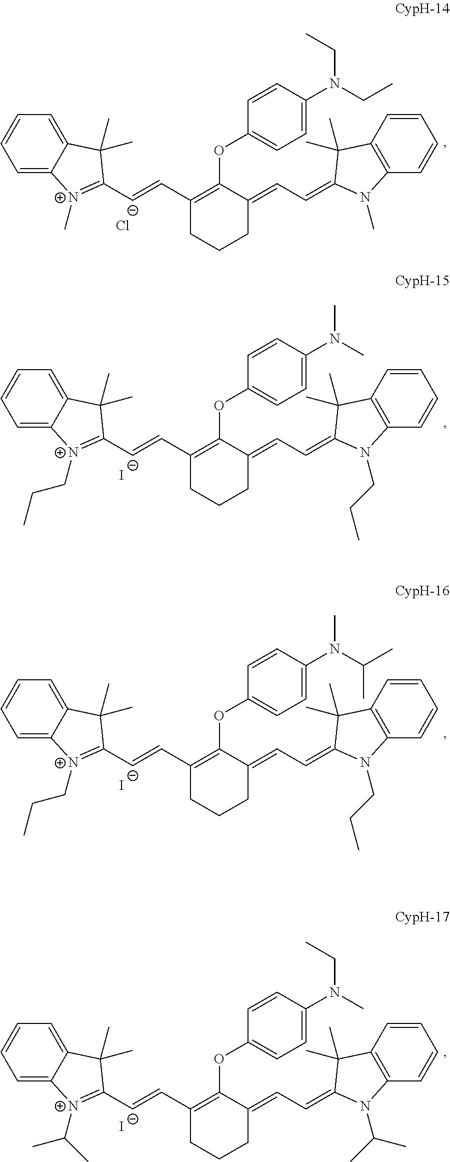

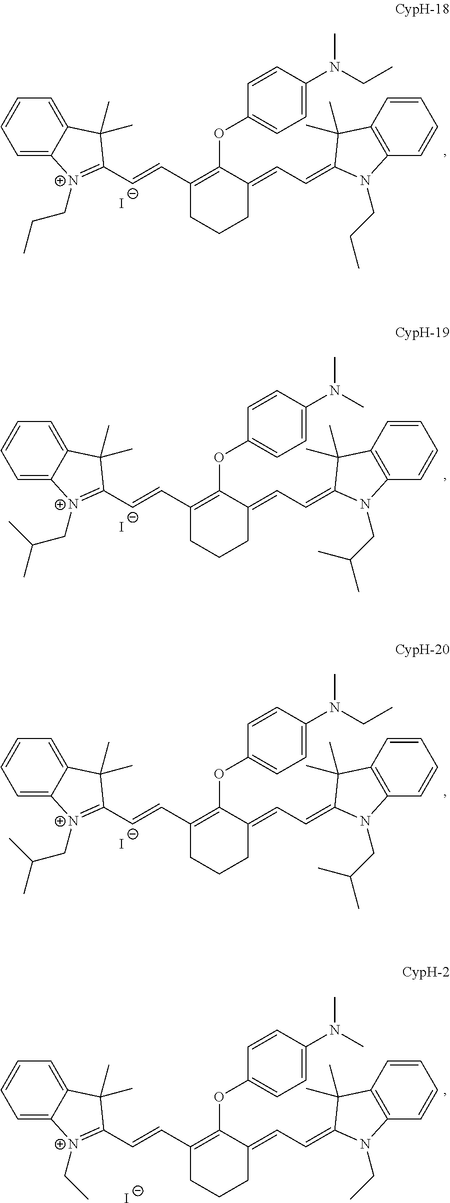

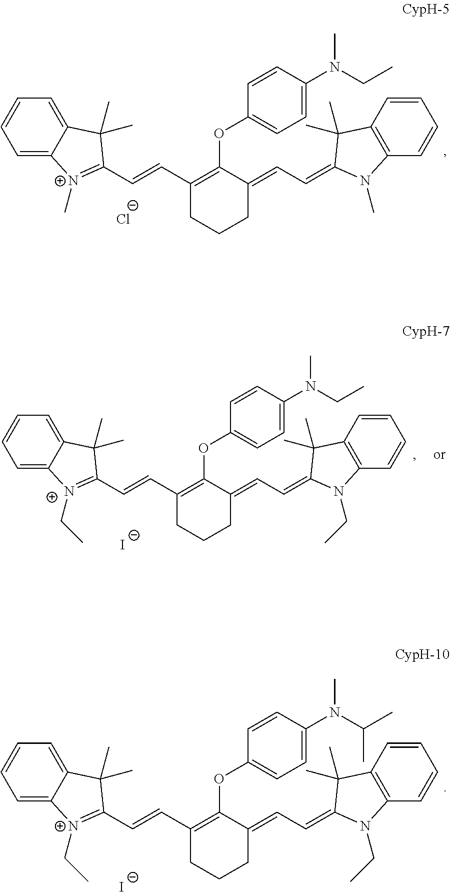 [SMILES: CCN(C)c1ccc(OC2=C(/C=C/C3=[N+](C)c4ccccc4C3(C)C)CCC/C2=C\C=C2\N(C)c3ccccc3C2(C)C)cc1 ; [Cl-] ; CCN(C)c1ccc(OC2=C(/C=C/C3=[N+](CC)c4ccccc4C3(C)C)CCC/C2=C\C=C2\N(CC)c3ccccc3C2(C)C)cc1 ; [I-] ; CCN1/C(=C/C=C2\CCCC(/C=C/C3=[N+](CC)c4ccccc4C3(C)C)=C2Oc2ccc(N(C)C(C)C)cc2)C(C)(C)c2ccccc21 ; [I-]]

In various examples, the compound has the following structure:

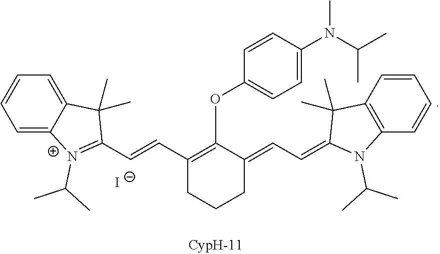 [SMILES: CC(C)N(C)c1ccc(OC2=C(/C=C/C3=[N+](C(C)C)c4ccccc4C3(C)C)CCC/C2=C\C=C2\N(C(C)C)c3ccccc3C2(C)C)cc1 ; [I-]]

Example B. A composition comprising a compound according to Example A and a pharmaceutically acceptable carrier. For example, the composition may have desirable permeation characteristics. Carriers with desirable permeation characteristics include, but are not limited to, propylene glycol, isopropanol, oleic acid, and polyethylene glycol analogs, and the like, and combinations thereof. It is desirable that the composition is non-lethal to cells. It is believed that osmolarity regulating agents may be used. Examples of osmolarity regulating agents include, but are not limited to, sugars (e.g., monosaccharides, such as, for example, glucose, fructose, sorbose, xylose, ribose, and the like, and combinations thereof, disaccharides, such as, for example, sucrose, sugar-alcohols, such as, for example, mannitol, glycerol, inositol, xylitol, adonitol, and the like, and combinations thereof, and amino acids, such as, for example, glycine, arginine, and the like and combinations thereof. Stabilizers may be used. Examples of stabilizers are known in the art. In various examples, the composition may comprise 0.5 to 10 μM compound, including every 0.01 μM value and range therebetween, in phosphate buffered saline with a pH of 6.5 to 7.5, including every 0.01 pH value and range therebetween, and 0.1 to 1.0% by volume DMSO, including 0.01% by volume value and range therebetween. In various examples, the composition may comprise 0.5 to 10 μM compound, including every 0.01 μM value and range therebetween, in phosphate buffered saline with a pH of 6.5 to 7.5, including every 0.01 pH value and range therebetween. The composition is a composition suitable for topical and/or oral administration (e.g., a sprayable composition).

Example C. A method of determining the presence and/or location of a solid tumor in an individual in need of treatment comprising: administering a compound according to Example A or a composition according to Example B to an area of interest on/in the individual; exposing the area of interest to electromagnetic radiation (e.g., light having a wavelength in the near infrared region (NIR) (e.g., 750 to 1500 nm)); and imaging and/or visualizing the area of interest, wherein the presence and/or location of the solid tumor is determined. After application of the compound or composition, a signal may be detected within several minutes (e.g., less than 5 minutes, less than 4 minutes, less than 3 minutes, less than 2 minutes, less than 1 minute, less than 55 seconds, less than 50 seconds, less than 45 seconds, less than 40 seconds, less than 35 seconds, less than 30 seconds, less than 25 seconds, less than 20 seconds, less than 15 seconds, less than 10 seconds, or less than 5 seconds). The fluorogenic signal will be developed in the neoplastic tumor tissue. Administration may occur by various non-intravenous delivery methods, such as topical administration (e.g., sprayed on the area of interest) or intraperitoneal delivery (i.p.). The administering may be a topical administration. The topical administration may be sprayed. In various examples, the topical administration is an oral rinse. In various examples, there is no washing prior to the imaging and/or visualizing. In various examples, the solid tumor is chosen from ovarian tumors, skin cancer, pancreatic cancer, genitourinary cancer, colon tumors, bladder tumors, brain tumors, esophagus tumors, cervical tumors, oral tumors, and the like, and combinations thereof. The application or administration to the solid tumor may result in the protonation of the compound. In various examples, the electromagnetic radiation is in the near-IR range.

Example D. A method of imaging a solid tumor comprising: applying or administering a compound according to Example A or a composition according to Example B to the solid tumor; exposing the area of interest to electromagnetic radiation; and obtaining an image of the solid tumor. After application of the compound or composition, a signal may be detected within several minutes (e.g., less than 5 minutes, less than 4 minutes, less than 3 minutes, less than 2 minutes, less than 1 minute, less than 55 seconds, less than 50 seconds, less than 45 seconds, less than 40 seconds, less than 35 seconds, less than 30 seconds, less than 25 seconds, less than 20 seconds, less than 15 seconds, less than 10 seconds, or less than 5 seconds). Administration may occur by various non-intravenous delivery methods, such as topical administration (e.g., sprayed on the area of interest) or intraperitoneal delivery (i.p.). The applying or administering is a topical application or topical administration. The topical administration may be spraying. The solid tumor may be chosen from ovarian tumors, skin cancer, pancreatic cancer, genitourinary cancer, brain tumors, colon tumors, bladder tumors, esophagus tumors, cervical tumors, oral tumors, and the like, and combinations thereof. The electromagnetic radiation may be in the near-IR range.

Example E. A method for determining margins of a solid tumor: administering a compound according to Example A or a composition according to Example B to an area of interest on/in the individual; exposing the area of interest to electromagnetic radiation (e.g., light having a wavelength in the near infrared region (NIR) (e.g., 750 to 1500 nm)); imaging and/or visualizing the area of interest, and determining the margin of the solid tumor. After application of the compound or composition, a signal may be detected within several minutes (e.g., less than 5 minutes, less than 4 minutes, less than 3 minutes, less than 2 minutes, less than 1 minute, less than 55 seconds, less than 50 seconds, less than 45 seconds, less than 40 seconds, less than 35 seconds, less than 30 seconds, less than 25 seconds, less than 20 seconds, less than 15 seconds, less than 10 seconds, or less than 5 seconds). The signal may be compared against the fluorescence signal of non-cancerous tissue in/of the area of interest to determine the margins of the tumor. Administration may occur by various non-intravenous delivery methods, such as topical administration (e.g., sprayed on the area of interest) or intraperitoneal delivery (i.p.). The applying or administering is a topical application or topical administration. The topical administration may be spraying. The solid tumor may be chosen from ovarian tumors, skin cancer, pancreatic cancer, genitourinary cancer, brain tumors, colon tumors, bladder tumors, esophagus tumors, cervical tumors, oral tumors, and the like, and combinations thereof. The electromagnetic radiation may be in the near-IR range.

Example F. A kit comprising a compound according to Example A or a composition according to Example B. A kit may comprise the compound (e.g., the compound as a lyophilized powder or film) and a pharmaceutically acceptable carrier. The two components may be mixed and sprayed onto a tissue of interest.

### EXAMPLE 1

The following example shows compounds of the present disclosure, as well as toxicity data and in vivo data for the compounds.

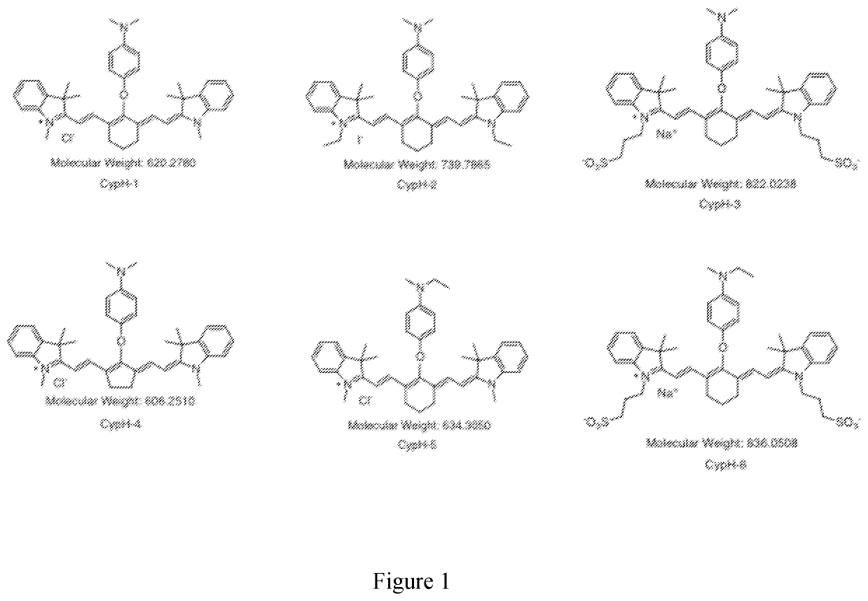

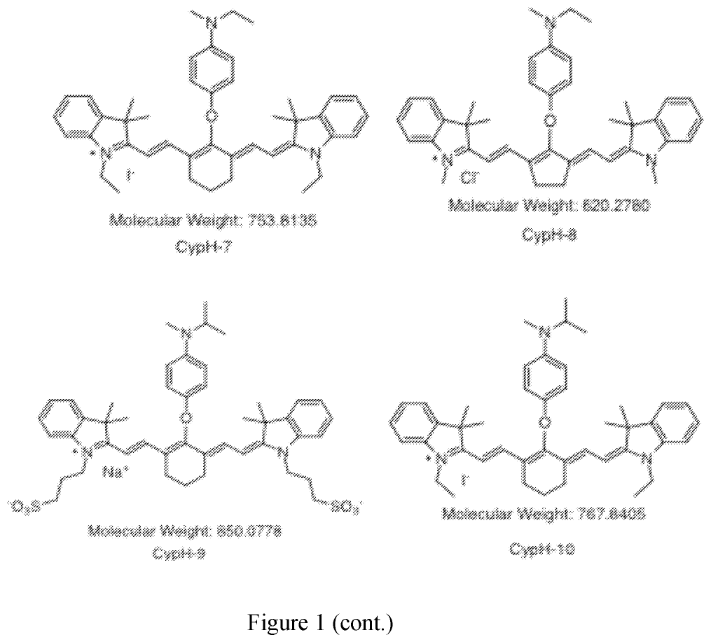

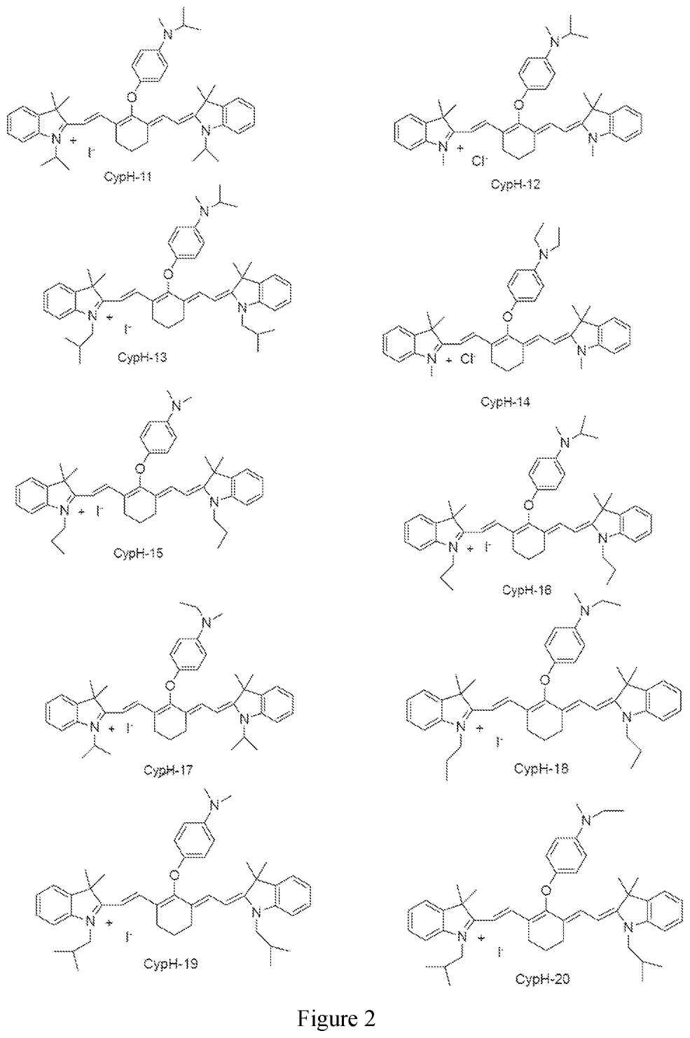

FIGS. 1 and 2 show compounds of the present disclosure.

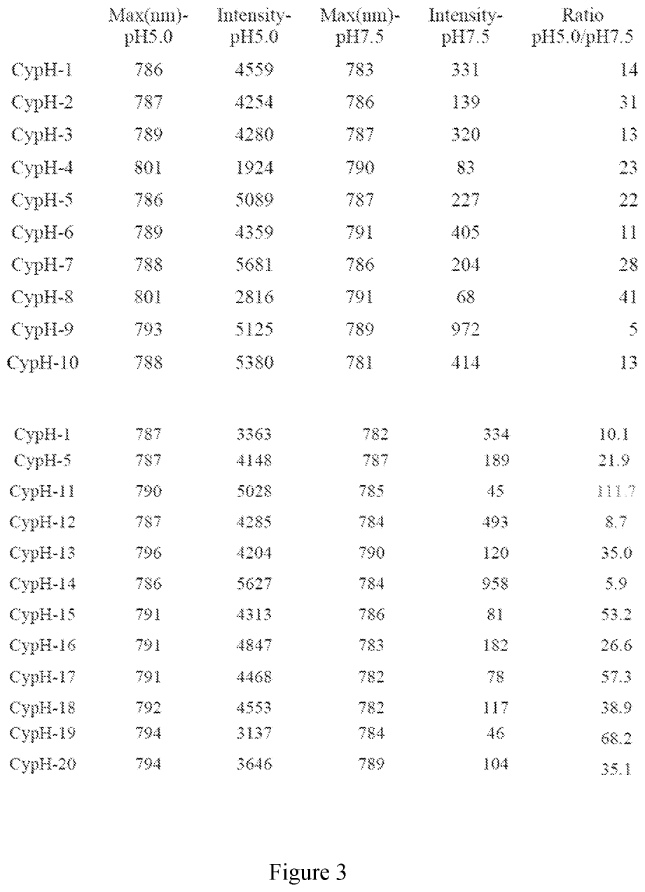

FIG. 3 shows emission maximum and intensity difference at pH 5.0 and 7.5.

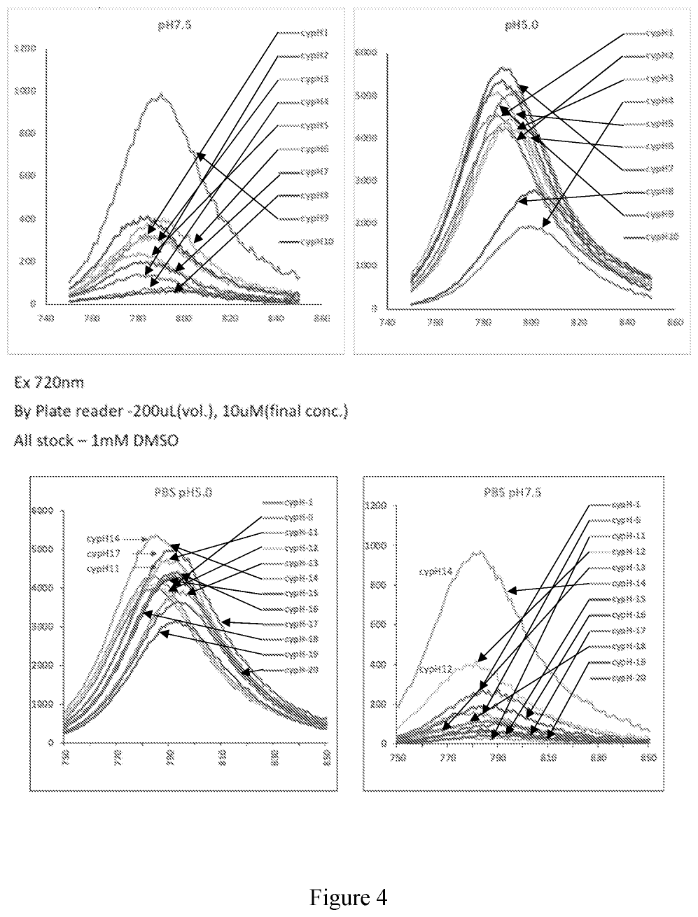

FIG. 4 shows fluorescence spectra of compounds of the present disclosure.

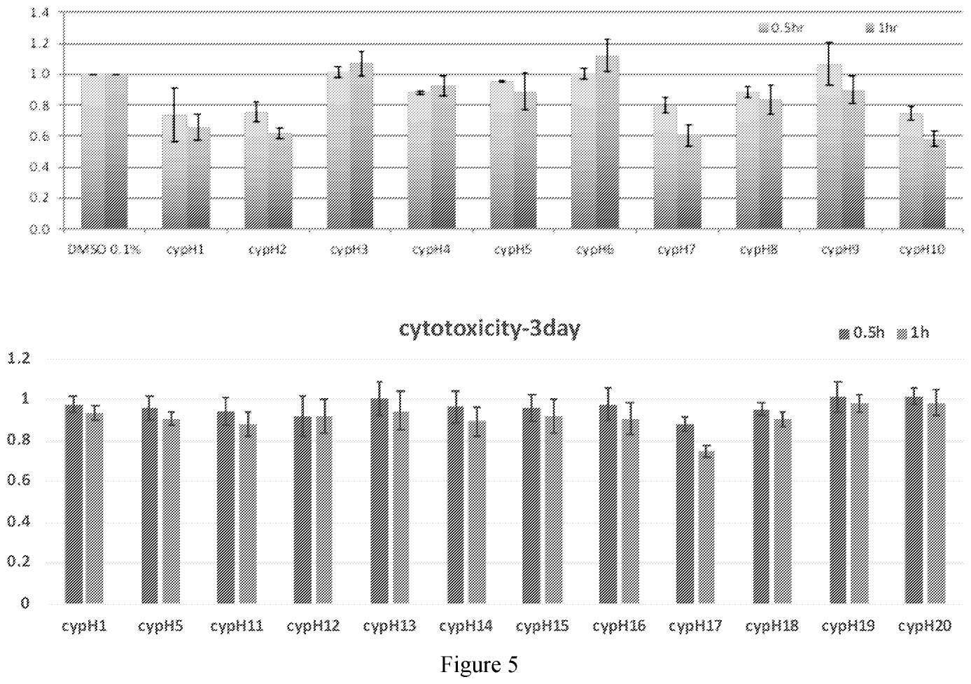

FIG. 5 shows cytotoxicity data of the compounds of the present disclosure. A CCK8 MMT assay was performed using 1 μM of each compound with 0.1% DMSO with RPMI. Cells were incubated for 0.5 or 1 hour, washed with fresh media, and then incubated for 3 days.

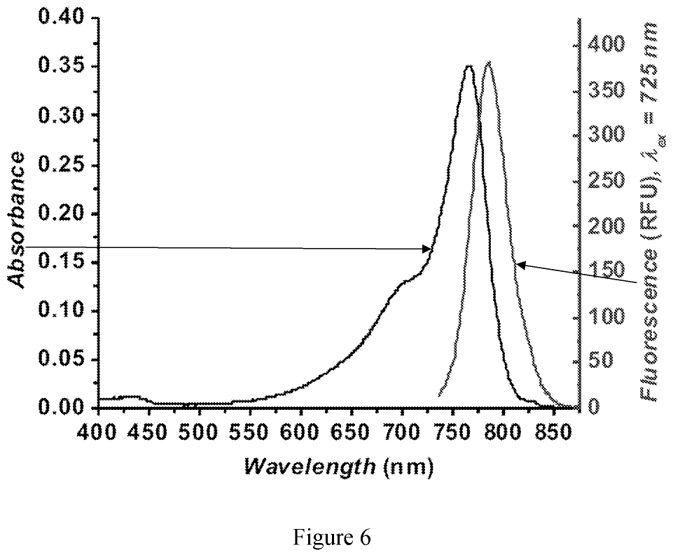

FIG. 6 shows absorbance and fluorescence spectra of CypH-11 (2 μM in pH 5.0 phosphate buffer solution. Exₘₐₓ=766 nm and Emₘₐₓ=785 nm.

In vivo imaging study. The animals inoculated with RFP-ovsaho cells in flank were used for the imaging study. To imitate the morphology of metastasized human ovarian cancer, the tumor size was controlled to 2 mm. The skin was removed prior to the spray of dye (2 μM, in saline). The tumor areas were sprayed and imaged using Cy7 filter set at different time points. RFP images were also acquired for tumor co-registration. The contrast ratio was calculated by [signal of tumor]/[signal of adjacent muscle].

Following the in vitro and cellular validation, we thought that the best candidate, CypH-11, which has excellent fluorescence property in different pH and in cells, could be an ideal agent to augment the detection of small cancerous lesions otherwise unnoticeable to surgeons. To verify the presumption, RFP positive ovsaho ovarian cancer cells were subcutaneously inoculated in mice. When tumors were about 2 mm in size, the skin was removed and the CypH-11 solution (2 μM in saline) was sprayed onto the tumor areas. A fluorescent signal highlighting the tumor quickly developed within a minute after the spray's application. The contrast continued to increase slightly and quickly reached plateau at ˜7 min. The tumor signal is about 150% higher than the adjacent muscle tissues, and the CypH-11 signal co-registered well with the RFP signal. Notably, no signal increase was observed in the normal tissues, indicating this CypH-11 signal enhancement is tumor specific. In a separate set of experiment, animals were inoculated with two tumors. Each tumor was sprayed either with the prototype dye CypH-1 or CypH-11, and imaged simultaneously. CypH-11 showed near instant signal enhancement, while the CypH-1 could only showed minimally contrast. This result strongly supported the benefit of our modification.

To further confirm the signal enhancement is pH dependent, a commercially available always-on dye Cy7 which has similar absorption and emission property, was applied to the tumor under the exact same condition. As expected, this pH independent Cy7 dye gave strong signal in all tissues. Totally different from CypH-11, Cy7 showed no appreciable differential contrast. Because of the fluorogenic property of CypH-11, the signal development could be imaged directly, without needing a washing step. These spray experiments suggested that a pH dependent CypH-11 could be used as an aerosol spray for real time tumor detection.

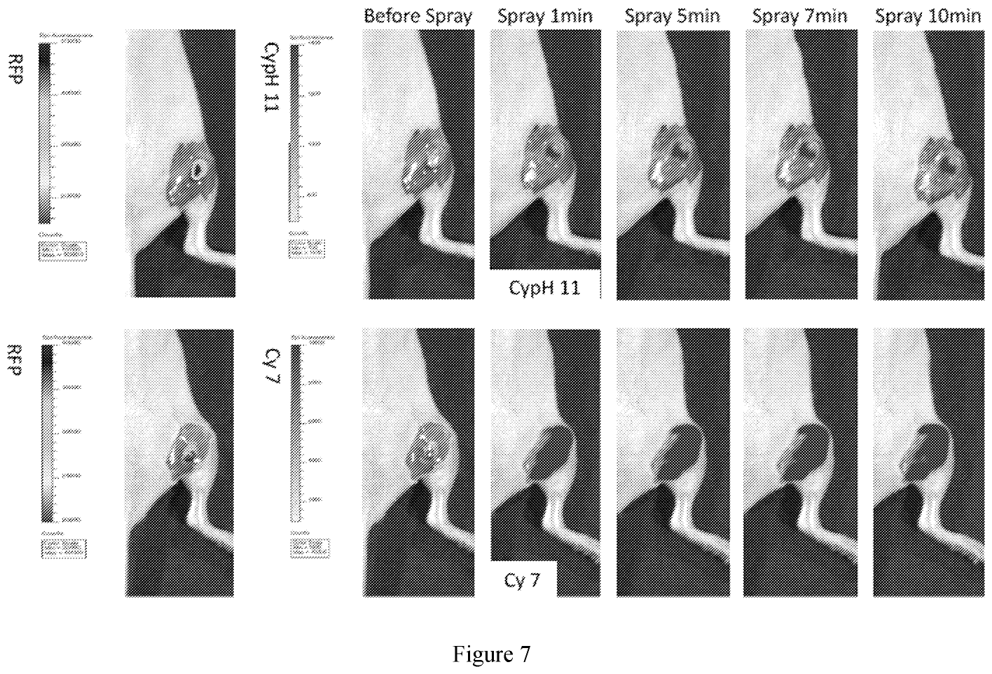

FIG. 7 shows a comparison of pH responsive CypH-11 and pH insensitive Cy7.

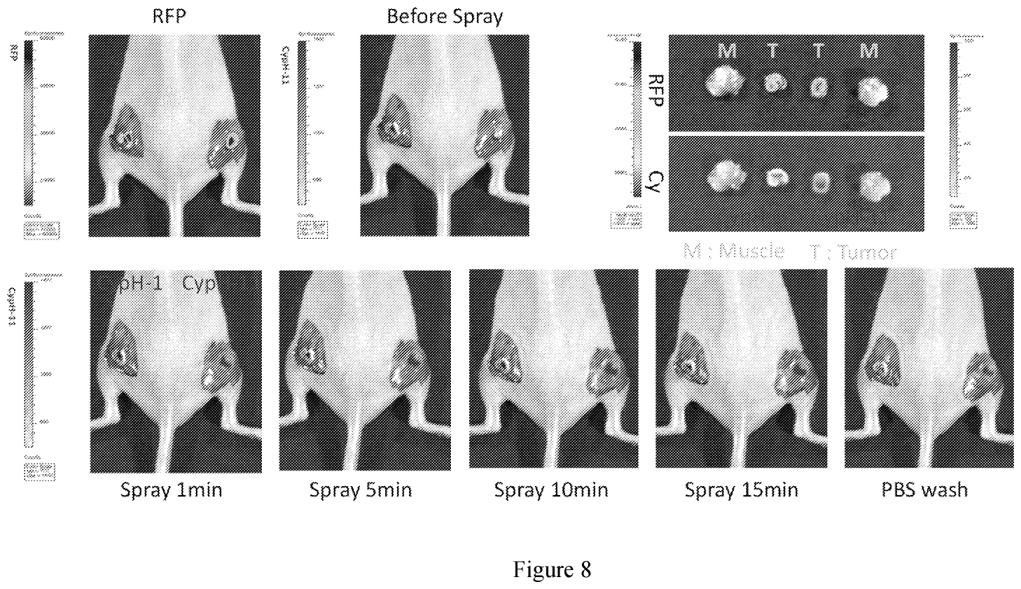

FIG. 8 shows a comparison of CypH-1 and CypH-11.

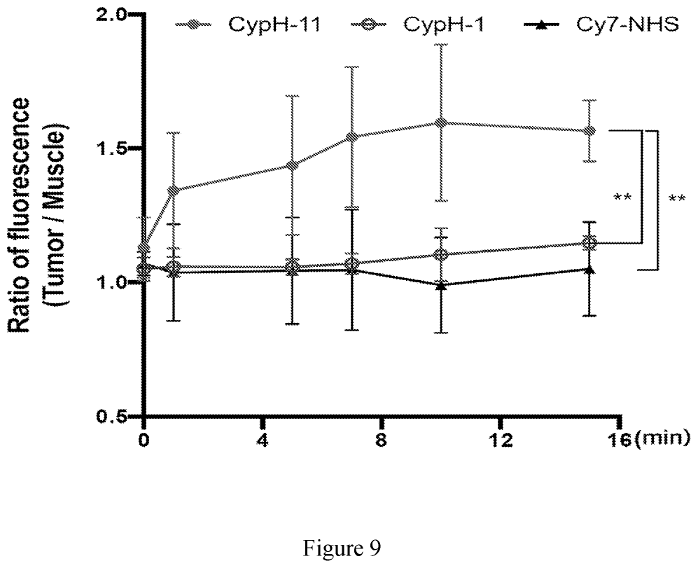

FIG. 9 shows tumor/muscle contrast ratio of CypH-11, CypH-1 and Cy7 at different time points.

### EXAMPLE 2

The following example shows compounds of the present disclosure and synthesis and characterization thereof.

Characterization: All new CypH dyes were characterized by proton NMR and mass spectrometry to confirm identity. NMR data was consistent with the structure and mass spectrometry results gave the expected mass of the dye±0.5 amu. An HPLC method was developed (see below) and used to assess dye purity. All dyes showed good purity of >95%. Retention times on the column generally correlated with lipophilicity of the dyes with water soluble dyes containing sulfate groups eluting out earlier (@10.6 min for CypH-3, 6, 9) than CypH-1 (@11.9 min) and the more lipophilic dyes later. Optical properties of the dyes, acid dissociation constants and solubilities were also determined. Characterization data is summarized in Table 1. Synthetic intermediates were also characterized by proton NMR.

The HPLC method used for dye purity consisted of a Phenomenex reverse phase Luna C8(2) column (5 μm, 100A, 250×4.6 mm, cat # 00G-4248-30) with solvents A (50% aqueous methanol+0.1% TFA) and B (100% methanol+0.1% TFA). Solvent gradient was 0-3min (0% B), 3-10 min (100% B), 10-20 min (100% B) and 20-25 min (0% B) with a flow rate of 1 mL/min. Detection was by photodiode array at absorbance maximum of the dye.

TABLE 1
Chemical properties of the CypH dyes.

| | | | | | | Ex Coeff | |
|---|---|---|---|---|---|---|---|
| | | | | | Abs. Max | (M⁻¹cm⁻¹) | |
| | | | | Retention | (nm) in | @ Abs | |
| CypH # | M⁺ calc. | M⁺ obs. | % purity | time | EtOH | Max | Solubility |
| 1 | | | 100 | 11.9 | 760 | 74,450 | >1 mM in |
| | | | | | | | EtOH |
| 2 | 612.4 | 612.5 | 99.0 | 12.3 | 764 | 139,100 | >1 mM in |
| | | | | | | | EtOH |
| 3 | 798.3 | 798.6 | 100 | 10.6 | 768# | 164,900 | >1 mM in |
| | | | | | | | H₂O |
| 4 | 570.3 | 570.5 | 99.3 | 12.4 | 775 | 58,000 | >1 mM in |
| | | | | | | | EtOH |
| 5 | 598.4 | 598.5 | 100 | 11.5 | 762 | 134,000 | >1 mM in |
| | | | | | | | EtOH |
| 6 | 814.4 | 814.6 | 100 | 10.6 | 768# | 150,400 | >1 mM in |
| | | | | | | | H₂O |
| 7 | 626.4 | 626.7 | 99.0 | 12.0 | 764 | 179,500 | >1 mM in |
| | | | | | | | EtOH |
| 8 | 584.4 | 584.5 | 95.6 | 11.8 | 777 | 72,700 | >1 mM in |
| | | | | | | | EtOH |
| 9 | 828.4 | 828.6 | 100 | 10.6 | 769# | 176,300 | >1 mM in |
| | | | | | | | H₂O |
| 10 | 640.4 | 640.4 | 97.9 | 12.0 | 765 | 175,400 | >1 mM in |
| | | | | | | | EtOH |
| 11 | 668.5 | 668.8 | 98.0 | 14.1 | 771 | 233,800 | >1 mM in |
| | | | | | | | EtOH |
| 12 | 612.4 | 612.2 | 97.6 | 13.6 | 767 | 255,600 | >1 mM in |
| | | | | | | | EtOH |
| 13 | 696.5 | 696.7 | 96.9 | 14.5 | 774 | 218,100 | >1 mM in |
| | | | | | | | EtOH |
| 14 | 612.4 | 612.3 | 97.6 | 13.7 | 767 | 215,800 | >1 mM in |
| | | | | | | | EtOH |
| 15 | 640.4 | 640.9 | 99.0 | 14.4 | 772 | 251,500 | >1 mM in |
| | | | | | | | EtOH |
| 16 | 668.5 | 668.8 | 99.5 | 14.2 | 771 | 248,000 | >1 mM in |
| | | | | | | | EtOH |
| 17 | 654.4 | 654.8 | 96.0 | 14.2 | 770 | 269,100 | >1 mM in |
| | | | | | | | EtOH |
| 18 | 654.4 | 654.9 | 96.6 | 14.3 | 772 | 257,300 | >1 mM in |
| | | | | | | | EtOH |
| 19 | 668.5 | 668.5 | 98.5 | 14.7 | 774 | 240,900 | >1 mM in |
| | | | | | | | EtOH |
| 20 | 682.5 | 682.6 | 98.6 | 14.5 | 774 | 218,600 | >1 mM in |
| | | | | | | | EtOH |

TABLE 2
Summary of Fluorescence Properties.

| | FL Max | FL | FL Max | FL | Ratio |
|---|---|---|---|---|---|
| CypH | (nm) | Intensity | (nm) | Intensity | Intensity |
| # | pH 5.0 | pH 5.0 | pH 7.5 | pH 7.5 | pH 5.0/7.5 |
| 1 | 787 | 3363 | 782 | 334 | 10.1 |
| 2 | 787 | 4254 | 786 | 139 | 30.6 |
| 3 | 789 | 4280 | 787 | 320 | 13.4 |
| 4 | 801 | 1924 | 790 | 83 | 23.2 |
| 5 | 786 | 5089 | 787 | 227 | 22.4 |
| 6 | 789 | 4359 | 791 | 405 | 10.8 |
| 7 | 788 | 5681 | 786 | 204 | 27.8 |
| 8 | 801 | 2816 | 791 | 68 | 41.4 |
| 9 | 793 | 5125 | 789 | 972 | 5.3 |
| 10 | 788 | 5380 | 781 | 414 | 13.0 |
| 11 | 790 | 5028 | 785 | 45 | 111.7 |
| 12 | 787 | 4285 | 784 | 493 | 8.7 |
| 13 | 796 | 4204 | 790 | 120 | 35.0 |
| 14 | 786 | 5627 | 784 | 958 | 5.9 |
| 15 | 791 | 4313 | 786 | 81 | 53.2 |
| 16 | 791 | 4847 | 783 | 182 | 26.6 |
| 17 | 791 | 4468 | 782 | 78 | 57.3 |
| 18 | 792 | 4553 | 782 | 117 | 38.2 |
| 19 | 794 | 3137 | 784 | 46 | 68.2 |
| 20 | 794 | 3646 | 789 | 104 | 35.1 |

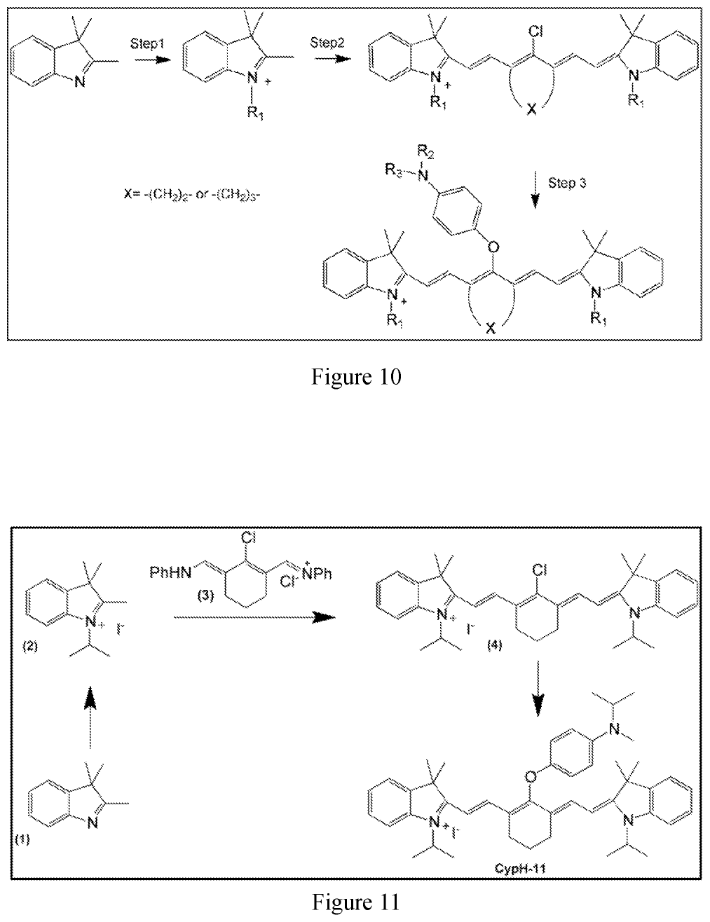

Structure Optimization: The new pH responsive dyes were modified from a previously published lead probe, CypH-1, which is a heptamethine cyanine dye that exhibits almost no fluorescence at neutral and basic conditions (≥7.0) but fluoresces in mildly acidic condition (≤pH 5.0). The excitation and emission maxima of CypH-1 are 760 and 777 nm, respectively. The signal intensity ratio of pH 5 over pH 7.5 was about 10. The meso-bridge ring size, lipophilicity and electro-density of CypH-1 was modified to provide better optical property. In round one screening from a library of 10 analogs, we found that the dyes with meso-cyclopentane rings have low fluorescence property at both pH 5 and pH 7.5 and the hydrophilic CypHs with —CH₂CH₂SO₃— substitution have good fluorescence property but low uptake on cell membranes. The best compound from this group was CypH-5 whose fluorescence ratio of pH 5.0/pH 7.5 was increased to 22. Based on this initial structure and property relationship, the second round of 10 new analogs were designed using CypH-5 as the core, focusing on modifying the electron density on the aniline ring and the indolinium ring. Various alkyl groups, such as methyl, ethyl, propyl, isopropyl and their combinations, were applied to these two positions. The general synthetic pathway is shown in FIG. 10.

The direct measurement of their fluorescence intensity showed that the CypH analogs (CypH-11 to 20) with methyl group at the indolinium ring had higher background fluorescence at pH 7.5. The CypH analogs with other longer alkyl chains had much lower background fluorescence. Their fluorescence ratio of pH 5.0/pH 7.5 was significantly improved from 10-20 to 50-110. Among them, CypH-11, which has a methyl isopropyl aniline and an isopropyl group on the indolinium ring, gave a 112-fold enhancement in fluorescence signal. CypH-11 has absorbance and emission maxima at 766 nm and 785 nm, respectively (FIG. 4) and was selected as the lead compound (vide infra).

Synthetic details and additional characterization of lead compound, CypH-11

CypH-11 was synthesized according to the scheme shown in FIG. 11 using the following experimental procedures.

Preparation of 4-(N-isopropyl, N-methyl)aminophenol starting material: 4-N-methylaminophenol (1.72 g, 0.01 mol), isopropyl iodide (1.70 g, 0.01 mol) and triethylamine 2.8 mL, 0.02 mol) are stirred in 10 mL of anhydrous chloroform at room temperature overnight. The solution is then concentrated, dissolved in a minimum volume of dichloromethane and purified by silica gel chromatography eluting with an increasing gradient of ethyl acetate in hexane (25-40% in 5% increments) to provide 4-(N-isopropyl, N-methyl)aminophenol (0.926 g, 56% yield). ¹H NMR (in d₆-DMSO): 8.60 (s, 1H, —OH), 6.67-6.61 (m, 4H), 3.80 (m, 1H), 2.50 (s, 3H, —CH₃), 1.02 (d, J=6.6 Hz, 6H, —(CH₃)₂).

Synthesis of N-isopropyl-2, 3, 3-trimethylindolinium iodide (2). 2,3,3-trimethylindoleine (1) (4 g, 0.025 mol) and 2-iodopropane (14 mL, 0.140 mol) are heated together at 140° C. for 72 h. Upon cooling the resulting thick oil is washed with diethyl ether to remove excess starting materials and the oil is then placed under high vacuum to remove remaining volatiles. The crude material (4.08 g, 49.6%) is analyzed by proton NMR and used in the next reaction. ¹H NMR (CDCl₃): 7.86-7.84 (m, 1H), 7.61-7.56 (m, 3H), 5.51 (m, 1H, N—CH), 3.31 (s, 3H, —CH₃), 1.92 (d, 6H, J=6.9 Hz, —(CH₃)₂), 1.67 (s, 6H, —(CH₃)₂)

Synthesis of 2-[2-[2-Chloro-3-[2-(1,3-dihydro-1-isopropyl-3,3-dimethyl-2H-indol-2-ylidene)-ethylidene]-1-cyclohexen-1-yl]-ethenyl]-1-isopropyl-3,3-dimethyl-3H-indolium iodide (4). N-[(3-(anilinomethylene)-2-chloro-1-cyclohexen-1-yl)methylene] aniline monohydrochloride (3) (0.281 g, 0.78 mmol, Millipore Sigma, St Louis) and N-isopropyl-2,3,3-trimethylindolinium iodide (0.575 g, 1.75 mmol) are heated at reflux in ethanol (20 mL) containing anhydrous sodium acetate (0.158 g, 1.93 mmol) for 3 h. The reaction mixture is concentrated and purified by silica gel chromatography eluting with an increasing amount of methanol in dichloromethane (2-5% in 1% increments) to provide (4) as a green solid (0.25 g, 48%). ¹H NMR (CDCl₃): 8.38 (d, J=14.0 Hz, 1H), 7.41-7.37 (m, 3H), 7.26 (m, 1H), 6.43 (d, J=14 Hz, 1H), 5.10 (m, 1H), 2.81 (t, J=6.2 Hz, 2H), 2.02 (m, 1H), 1.80-1.60 (m, ˜12H).

Synthesis of CypH-11. A solution of 4-(N-isopropyl, N-methyl)aminophenol (0.081 g, 0.491 mmol) in anhydrous N,N-dimethylformamide (5 mL) is stirred at RT and sodium hydride (0.022 g, 60% in oil, 0.55 mmol) is added followed by stirring for another 15 minutes to form the sodium phenoxide salt. Dye (2) (0.150 g, 0.225 mmol) is then added and the mixture stirred at RT overnight. The DMF is removed under vacuum and the residue dissolved in a small volume of dichloromethane and purified by silica gel chromatography eluting with an increasing gradient of methanol in dichloromethane (0-6% in 1% increments) to furnish CypH-11 as a green solid (51.0 mg, 28.5%). ¹H NMR (500 MHz, CDCl₃) δ 7.99 (d, J=14.1 Hz, 2H), 7.33-7.24 (m, 6H), 7.20-7.13 (m, 2H), 6.95 (d, J=9.1 Hz, 2H), 6.82 (d, J=9.1 Hz, 2H), 6.21 (d, J=14.1 Hz, 2H), 4.96-4.87 (m, 2H), 3.94-3.86 (m, 1H), 2.76 (t, J=6.0 Hz, 4H), 2.63 (s, 3H), 2.10-2.00 (m, 2H), 1.66 (d, J=7.0 Hz, 12H), 1.35 (s, 12H), 1.07 (d, J=6.6 Hz, 6H). ¹³C NMR (126 MHz, CDCl₃) δ 171.81, 165.23, 152.98, 146.18, 142.71, 141.85, 141.05, 128.22, 124.54, 123.07, 122.26, 116.84, 114.98, 112.76, 100.48, 50.97, 48.94, 48.75, 30.63, 28.19, 24.77, 21.22, 19.72, 18.83.

### EXAMPLE 3

The following example shows compounds, synthesis and characterization thereof, and methods of the present disclosure.

A near-infrared pH-responsive fluorogenic dye, CypH-11, was designed to be used as a sensitive cancer spray to highlight cancerous tissues during surgical operations, minimizing the surgeon's subjective judgment. CypH-11, pKa 6.0, emits almost no fluorescence at neutral pH, but fluoresces brightly in an acidic environment, a ubiquitous consequence of cancer cell proliferation. After topical application, CypH-11 was absorbed quickly, and its fluorescence signal in the cancerous tissue was developed within a minute. The signal to background ratio was 1.3 and 1.5 at 1 and 10 min, respectively. The fluorogenic property and near-instant signal development capability enable the “spray-and-see” concept. This fast-acting CypH-11 spray could be a handy and effective tool for fluorescence guided surgery, identifying small cancerous lesions in real-time for optimal resection without systemic toxicity.

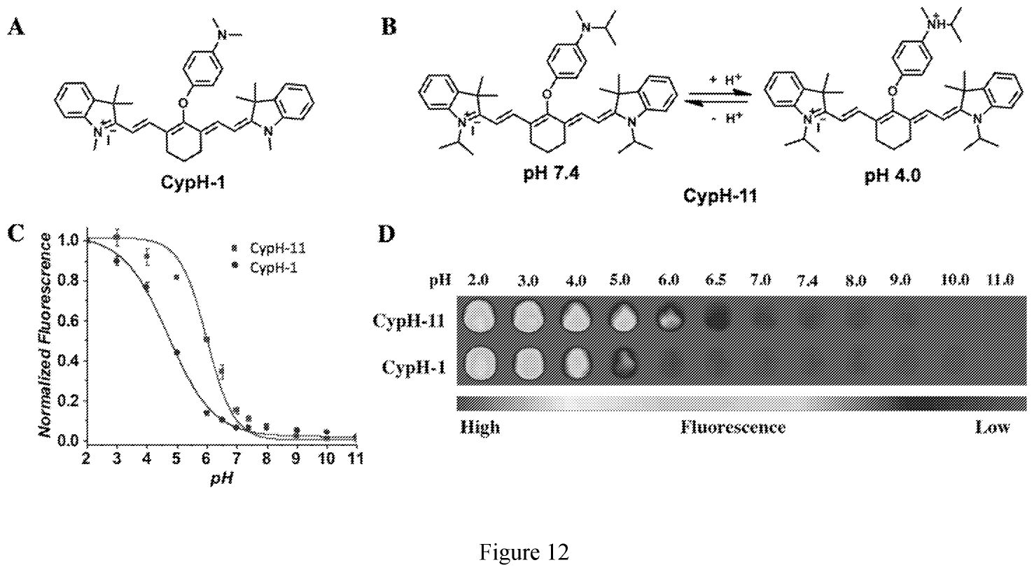

Design and characterization of pH-responsive fluorogenic CypH-11. Acidic pH in the tumor microenvironment, caused by enhanced glycolysis, is a widely used target for tumor diagnosis and therapy development. A pH-responsive fluorescent dye, CypH-1, was previously made by installing a pH-sensitive amino moiety onto a NIR cyanine fluorophore (FIG. 12A). At physiological pH (pH=7.4), the dimethylamino phenol group is not protonated and CypH-1 exhibited an extremely low fluorescence due to the photo-induced electron transfer (PeT) quenching. Whereas in acidic conditions, the amino group on CypH-1 is protonated and PeT quenching is blocked, resulting in a high fluorescence recovery (FIG. 12). When tested in a murine ovarian cancer model by IP injection, CypH-1 showed significantly higher fluorescence in the tiny lesions than the normal tissues, but it failed to detect the ovarian tumors by spraying, probably due to its low pKₐ (pKₐ=4.7). Thus, the pKₐ of this fluorogenic probe was not suitable for optimal imaging by spraying. Considering that the pH in the tumor is around 6.2-6.9 and the pH in normal tissue is 7.4, a fluorescent probe that has a pKₐ close to the 6.2-6.9 window would be preferred. Therefore, the 4-(dimethylamino)phenol moiety of CypH-1 were substituted with a more electron-rich 4-(N-isopropyl, N-methyl)aminophenol group, and the methyl groups on the indolinium rings were replaced with election donating isopropyl groups to provide a more optimized dye, CypH-11 (FIG. 12B).

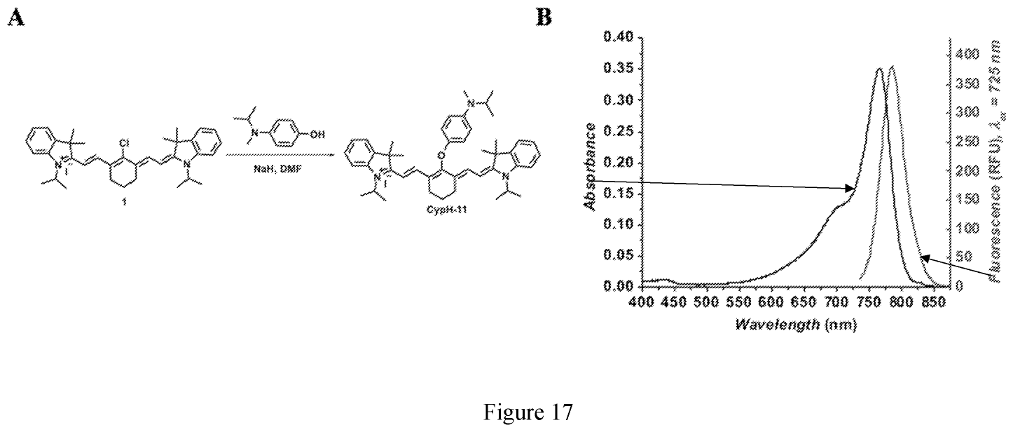

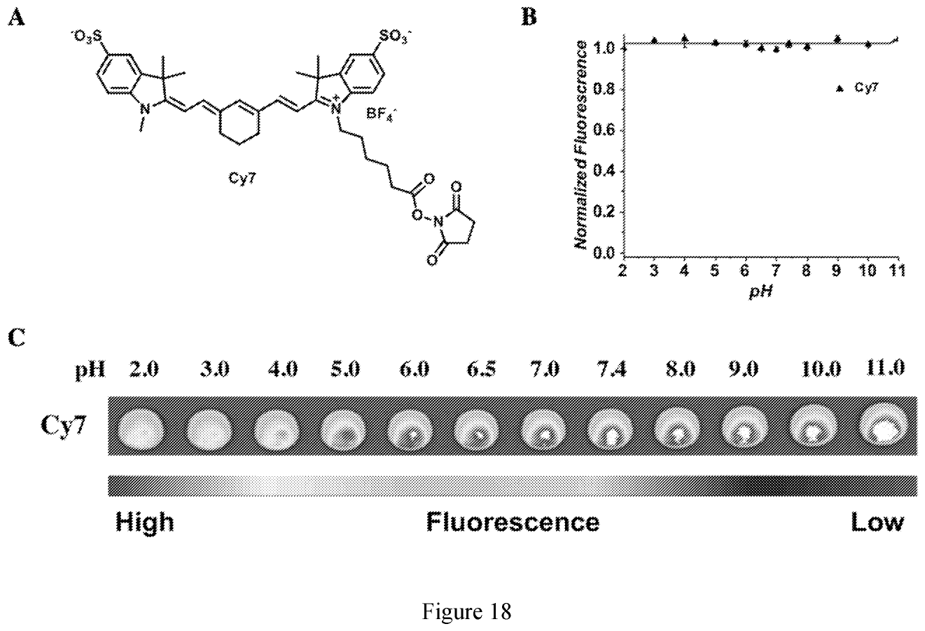

CypH-11 was synthesized by reacting 4-(N-isopropyl, N-methyl)aminophenol with fluorophore 1 under basic conditions (FIG. 17A). The respective absorption and emission peaks were centered at 765 nm and 785 nm (FIG. 17B), respectively, and the quantum yield (Φ) of CypH-11 at pH 4.0 was 3.3%. As designed, CypH-11 showed a notable higher pKₐ value (pKₐ=6.0) than CypH-1 (pKₐ=4.7). Both CypH-11 and CypH-1 showed low fluorescence in neutral and basic solutions, but strong fluorescence in acidic solutions (FIGS. 12C and 12D). The titration curves and images in different pH solutions confirmed that CypH-11 is more sensitive to pH variations in the physiological environment, pH 5.0-7.0. A commercially available pH-insensitive fluorophore, Cy7, which has similar excitation and emission wavelengths, was included as a reference in biological studies. Cy7 fluoresced brightly at all pH values and exhibited no pH-dependency (FIG. 18).

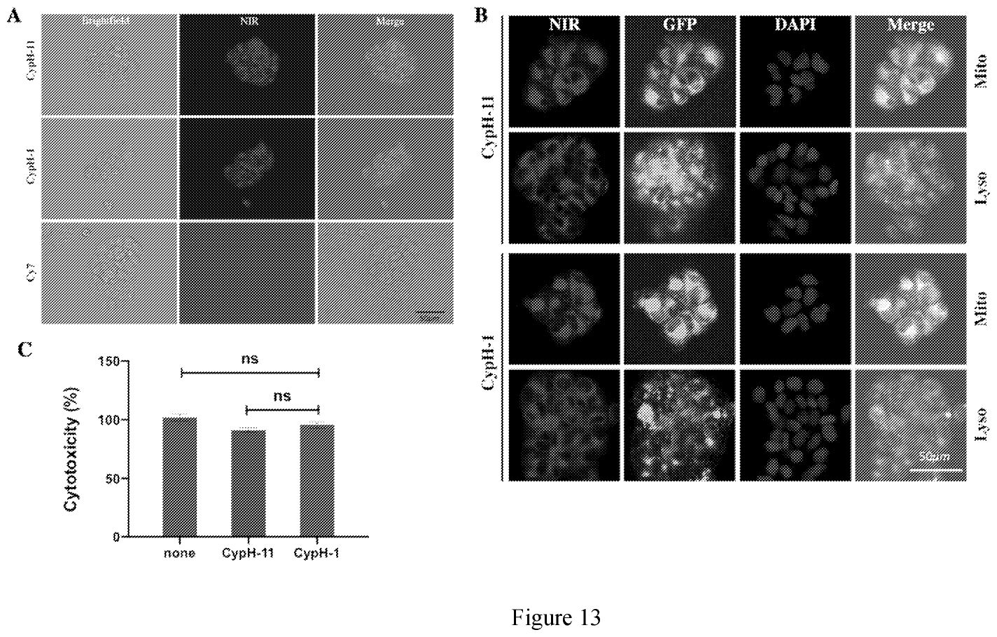

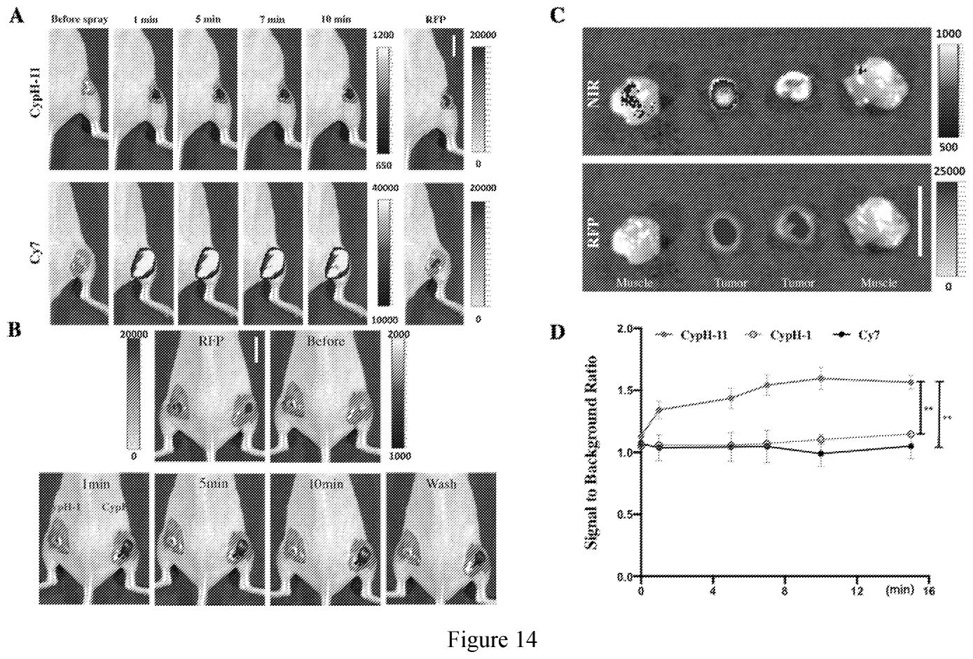

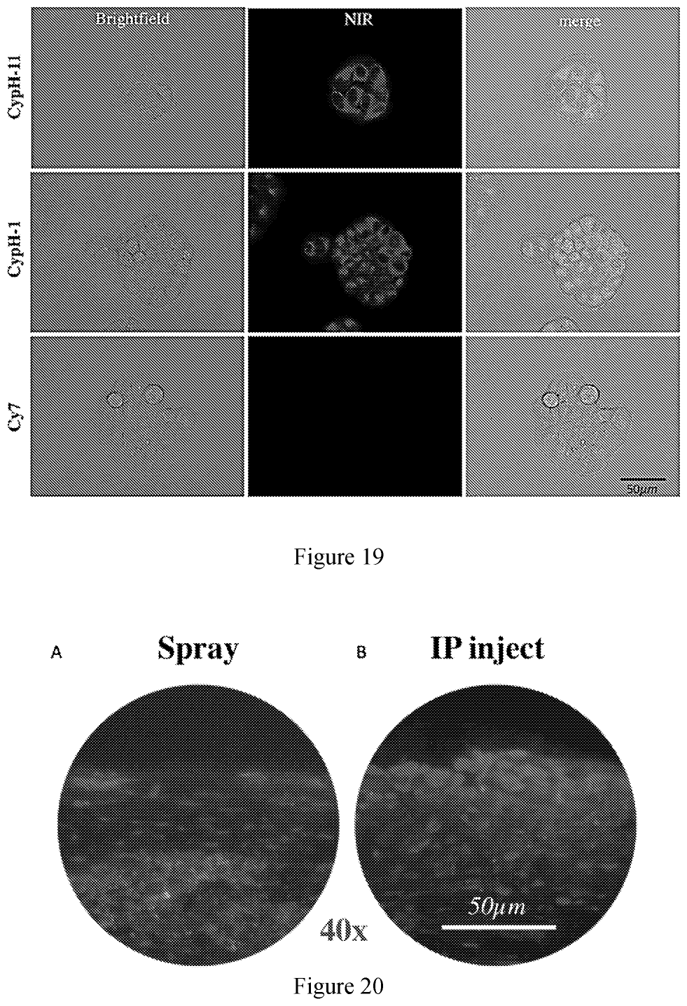

Evaluation of CypH-11 in cancer cells. An ovarian cancer cell line, OVASAHO, was used to evaluate the performance of CypH-11, CypH-1, and Cy7. OVASAHO cells were incubated with the respective probe (2 μM) for 1 hr, and the cellular fluorescence images were captured in the presence of the dye-containing medium (FIG. 13A). Strong intracellular fluorescence and low medium fluorescence were observed in the CypH-11 and CypH-1 treated wells, but the Cy7-treated well showed over-saturated fluorescence. This distinct difference is because of the pH sensitivity of CypH-11 and CypH-1. Both of them have low fluorescence in the cell culture medium (pH=7.4), but their fluorescence is turned on upon entering the cellular acidic compartments. In contrast, Cy7 showed constant high fluorescence in the physiological pH range. After washing with a fresh culture medium, the cell images were captured again (FIG. 19). Both CypH-11 and CypH-1 treated cells showed high fluorescence indicating their significant cellular penetration and retention, but very dim fluorescence was observed for Cy7 indicating its poor cellular penetration or retention. The intracellular distribution of dyes was investigated by co-staining with mitochondrial, lysosomal and nuclear trackers (FIG. 13B). Both CypH-11 and CypH-1 showed much better overlap with the mitochondria tracker (Pearson values: 0.70 and 0.90 respectively) than with the lysosome tracker. Furthermore, cytotoxicity studies with OVASAHO cells showed that treatment with 2 μM CypH-11 or CypH-1 for 1 hr does not significantly impact cell viability, 91.0±4.6% and 95.5±6.9%, respectively (FIG. 13C).

Detection of subcutaneous tumors by spraying. To evaluate the in vivo performance of the fluorogenic probes via the “spray-and-see” technique, an OVASAHO subcutaneous tumor model was used. For a facile signal co-registration, the cells were first engineered to express red fluorescence protein (RFP). Two weeks after subcutaneous inoculating of OVASAHO/RFP-Luc cells into both flanks, the tumors reached around 5 mm in size. The skin on the tumor area was removed, then the CypH-11 or Cy7 solution (2 μM in PBS) was sprayed once onto the exposed area. The whole body fluorescence images were continuously captured at various time points without washing (FIG. 14A). The tumor tissues were conveniently delineated by their intrinsic RFP fluorescence. NIR fluorescence generated by CypH-11 exhibited a near-instant development (<1 min) in the tumor area but not in the neighboring normal area, and reached its plateau within 10 min. In contrast, Cy7 showed strong fluorescence in the whole sprayed areas due to its pH insensitive “always-on” nature.

CypH-11 and CypH-1 were sprayed on either side of the tumor for a side-by-side comparison. CypH-11 showed high fluorescence only on tumors but not on normal tissue, while CypH-1 showed very poor fluorescence enhancement in both the tumor and neighboring normal tissues (FIG. 14B). The sprayed area was then washed with PBS, and the signal was found to remain in the tumor, indicating that CypH-11 was absorbed by the tumor tissue and the signal was developed from within (FIG. 14B). Similar results were observed on the resected tumors and the neighboring normal tissues (FIG. 14C). The tumor-to-muscle signal ratios of these three dyes were plotted versus time (FIG. 14D). Immediately after spraying CypH-11, a significant level of fluorescence signal was detected in the tumor, and the signal continued to increase until 10 min. The signal to background ratio at 1, 5, and 10 min were 1.3, 1.4, and 1.5 respectively. In contrast, the tumor to muscle ratios of dark CypH-1 and always-on Cy7 remained around 1.0, indicating their inability of detecting tumors.

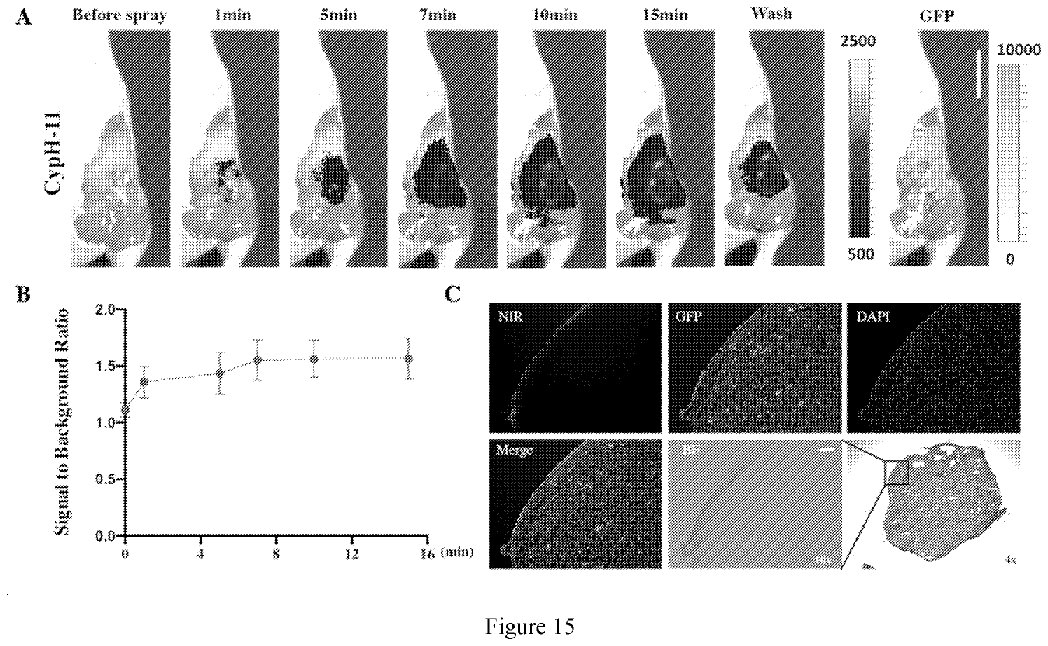

CypH-11 was further evaluated on a second subcutaneous tumor model, SKOV3/GFP-Luc, to confirm that OVASAHO tumor staining was not a single incidence. After spraying CypH-11 on the surgical area, a rapid signal development around the tumor was observed (FIG. 15A). Compared with the GFP signal, which indicated the exact position of the tumor, the NIR signal was highest on the border of the SKOV3 tumor. Interestingly, the pattern of the signal was different from the one seen in the OVASAHO tumor whose signal was quite consistent over the area. The increasing signal trend in the SKOV3 tumor was similar to that seen in the OVASAHO tumor (FIG. 15B), the signal to background ratio reached 1.3, 1.4, and 1.6 at 1, 5, and 10 min, respectively. As before, a PBS washing could not wash away the fluorescence signal, supporting the internalization of the sprayed CypH-11 (FIG. 15A).

To evaluate the CypH-11 distribution in tumors after spraying, the tumors were resected and sectioned into 14-μm-thick slides. The slides were stained with H&E and DAPI nucleus stain. Under a fluorescence microscope, GFP and DAPI fluorescence signals uniformly distributed across the tumor, but the CypH-11 signal was mainly on the outer layer of the tumor (FIG. 15C). The high magnification image showed the NIR signal depth was about 2-3 layers of cells (FIG. 20A). This shallow surface penetration could be attributed to the short and limited contract with the sprayed CypH-11.

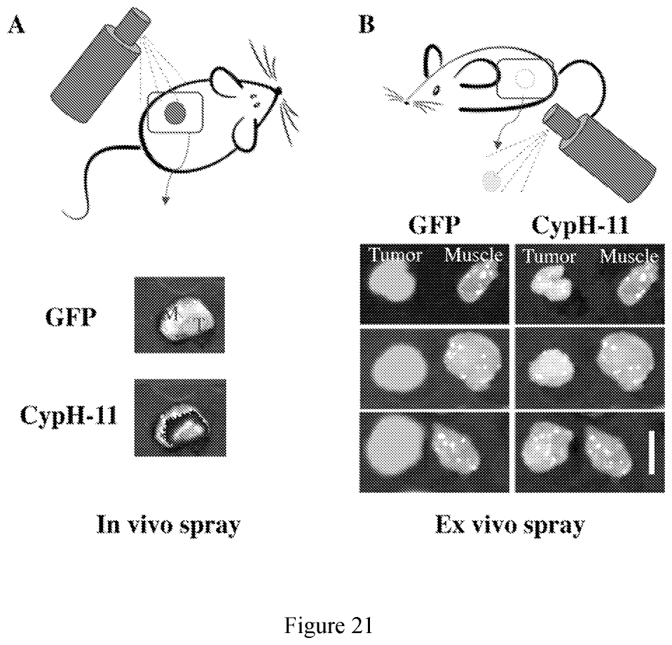

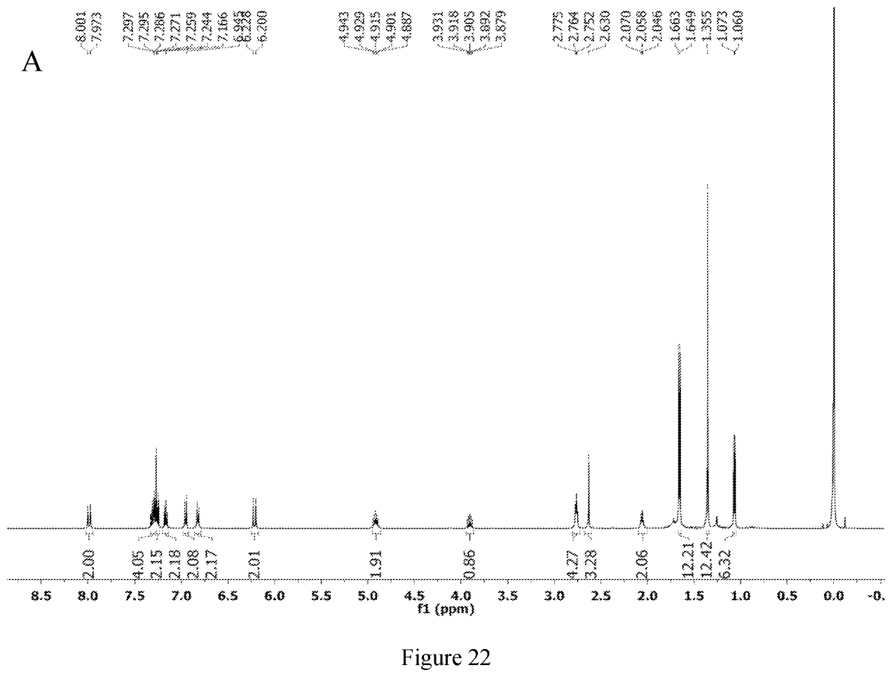

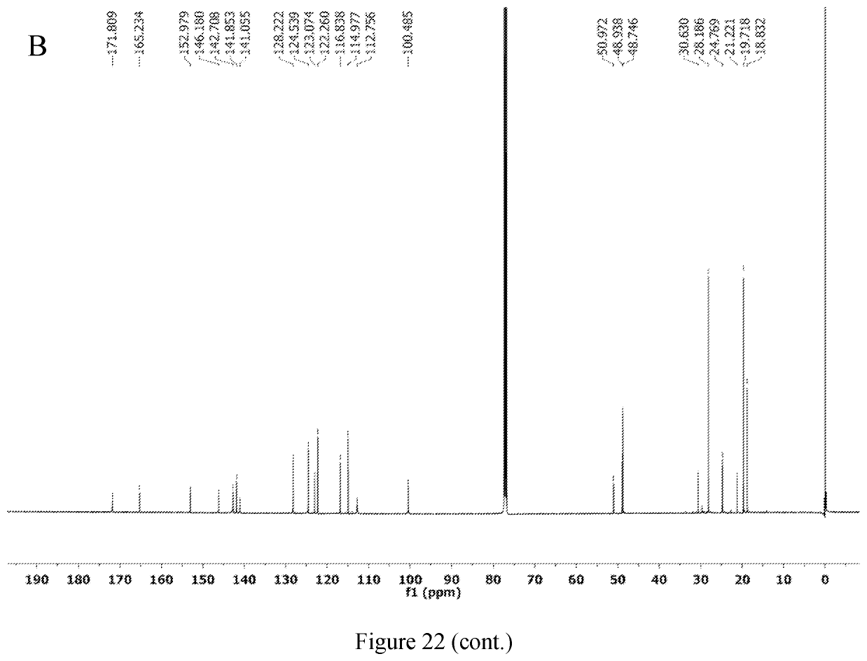

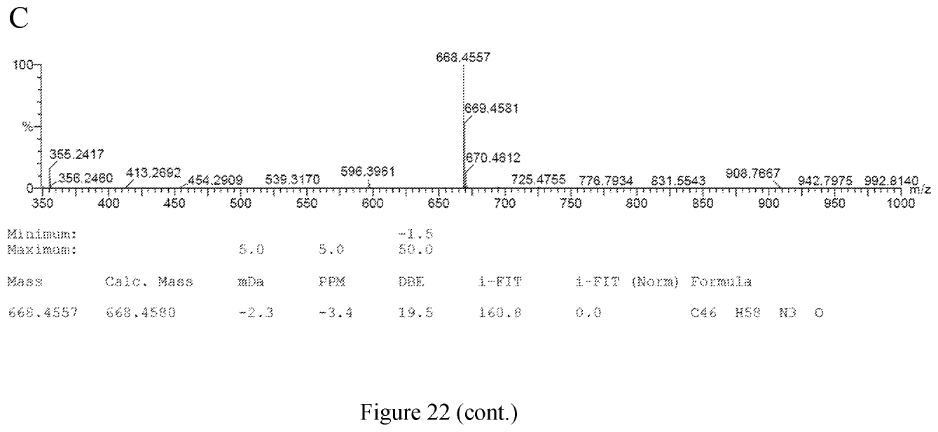

The usage of CypH-11 was explored on collected tissues. If successful, using CypH-11 could be applied to post-surgical tissue evaluation. When CypH-11 was sprayed onto tissues in living animals, the signal quickly developed and stayed in the tumor (FIG. 21A). The signal in the excised tissues could be detected weeks to months after storage. Conversely, when the tumor and muscle tissues were collected first, and then CypH-11 was sprayed onto these 20-min old “dead” tissues, no NIR signal could be detected (FIG. 21B), indicating only the live tissues can absorb and convert the topically applied fluorogenic CypH-11.

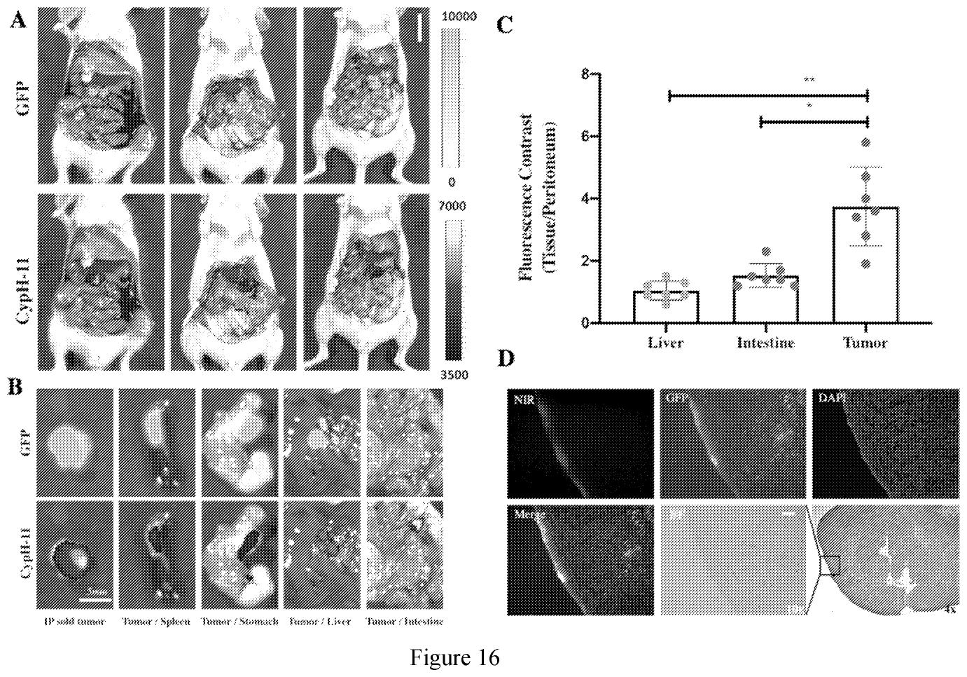

Detection of tiny disseminated ovarian tumors by intraperitoneal delivered CypH-11. Following the promising local “spray-and-see” application of CypH-11, it was also of interest to know whether this fast responsive fluorogenic dye could be used for rapid detection of intraperitoneally disseminated small ovarian tumors. To imitate peritoneally disseminated ovarian cancer, SKOV3/GFP-Luc cells were directly injected into the abdominal cavity of a mouse and tumor growth was followed by monitoring D-luciferin-induced bioluminescence. It took about two weeks to reach a strong bioluminescence signal, indicating tumor growth. CypH-11 (2 μM, 200 μL in PBS) was administered intraperitoneally, and an hour later the abdominal cavity was surgically exposed, and the bright field and NIR fluorescence images were immediately captured without washing. The GFP signal indicates the tumor location, and the NIR fluorescence is produced by CypH-11 (FIG. 16A). Because of the CypH-11's fluorogenic nature, the background signal was very low in normal tissues and organs so that a washing step was not needed. Excellent overlap between the CypH-11-generated fluorescence and the tumor GFP signal was observed. Following whole body imaging, which can only report large surface tumors (>3 mm) on the abdominal cavity, the tissues and the major organs (spleen, stomach, liver, and intestine) were collected to identify small and barriered tumors. The zoom-in view also showed a desirable correlation between tumor and CypH-11 signals (FIG. 16B). All tumors of variable sizes were highlighted and there was a 3- to 4-fold higher signal than the background of the peritoneum, liver, and intestine (FIG. 16C). More importantly, tumors as small as 1 mm, which pose a great challenge for a surgeon to remove, were clearly detected. The histological analysis also showed that CypH-11 was mainly in the outer layer of the tumors, but the signal was down to 6-7 layers of cells within an hour (FIGS. 16D and 20B). The deeper CypH-11 penetration observed here compared to its application by spraying is probably due to longer contact with a larger volume of the CypH-11 solution.

### Discussion

FGS is an up-and-coming technology because of its real-time visualization capability. Assisted by a tumor-specific fluorescence probe, under an exciting light, surgeons are able to “see” the fluorescent tumor through a video camera. A topical sprayable probe could be extremely valuable during the surgical procedure, especially for small tumor identification and tumor margin confirmation. When needed, the probe could be sprayed onto suspicious areas to highlight if any cancerous tissue is present for resection or if it is normal tissue to be avoided thereby improving safety. Recently, at least two topical agents, β-galactosidase sensitive SPiDER-βGal and γ-glutamyltranspeptidase sensitive gGL-HMRG have been reported to detect tumors, but their applications are limited to tumors expressing the targeted enzymes. To obtain a universal “spray-and-see” probe for instantaneous tumor visualization, the probe should target a general cancer hallmark and the signal conversion should be tumor-specific and near-instant. Since tumor acidosis is a ubiquitous consequence of cancer cell proliferation and growth, and the protonation reaction is instantaneous, a tumor pH-sensitive fluorogenic CypH-11 was designed to image cancerous tissues without requiring washing away excess dye.

CypH-11 is derived from a previously developed NIR cyanine dye, CypH-1. Although CypH-1 was pH-responsive, its pKa was not optimized for the pH in the tumor environment. Without intending to be bound by any particular theory, a dye whose pKa was closer to the pH of the tumor environment would be an improved dye for tumor detection. The introduction of electron-donating groups raised the pKa of CypH-11 to 6.0. Under basic conditions, the fluorescence is quenched due to the PeT effect between the lone pair electrons on the isopropyl-methyl amino group and the cyanine backbone of CypH-11 (FIG. 12B). Under acidic conditions, the amino group is protonated and the lone pair electrons are masked, resulting in a strong fluorescence signal. Measurement of probe fluorescence output in solution showed that the normalized fluorescence of CypH-11 at pH 6.0-6.5 is about 3.4 fold higher than that of CypH-1 (FIGS. 12C and 12D).

The cell imaging experiment confirmed the benefit of the fluorogenic property (FIG. 13). Both CypH-11 and CypH-1 gave a very low background signal in neutral culture medium (pH=7.4), so their distribution into intracellular acidic compartments was clearly visualized, without a washing step, whereas the highly fluorescent pH insensitive Cy7 overpowered everything both in cell culture and in vivo. CypH-1 showed minimum signal enhancement when sprayed onto the tumor area probably due to its low pKa resulting in ineffective fluorescence turn on. In contrast, CypH-11 highlighted the tumor and delineated the tumor margin with minimal background signal (FIGS. 14 and 15). Because the protonation step is almost an instantaneous reaction, the CypH-11 fluorescence turn-on in tumors is rapid (<1 min) requiring almost no waiting time. The ability to immediately visualize fluorogenic conversion in situ is a critical feature for a spraying agent.

Previously, it was demonstrated that an IP administered polymer-based protease activatable probe produced better detection of small-sized ovarian tumors compared with an IV administered one, and that IP administered CypH-1 was effective in detecting small tumors. In this study, it was shown that CypH-11, injected IP, was able to label very small ovarian tumors (<1 mm) within an hour and no washing step was needed before imaging (FIG. 16). Based on this fast-response rate and tumor selectivity, IP delivered CypH-11 may be easily translated into the clinic for optimal cytoreduction.

The CypH-11 fluorogenic signal development in the tumor is by direct contact with the tumor tissues. Due to the topical spray delivery and limited probe solution, it is reasonable that the NIR signal was restricted to the top layers of cells (FIG. 20). As acidic pH is a universal cancer marker, the pH-sensitive spray technology could be useful for many types of superficial tumors such as ovarian, cervical and colon cancers. A study of stage III or IV ovarian cancer patients treated with maximal cytoreduction (no gross residual lesions) demonstrated that each 10% increase in optimal cytoreduction was associated with a 5.5% increase in median survival. A more than 13 month longer median survival has been reported in patients with no residual tumor after optimal cytoreduction compared to those with residual tumor, suggesting that complete surgical cytoreduction is the most important prognostic indicator for survival. Sadly, current surgery is inadequate because 40% of the time it does not remove all tumor and residual microscopic and undetected tumor nodules. A spray agent, like CypH-11, that enhances the surgeon's ability to visualize then resect disease tissues during intraoperative procedures can be impactful.

### Conclusion

CypH-11 is a simple pH-sensitive fluorophore which exhibits negligible fluorescence at neutral pH but rapid bright fluorescence is turned-on under mildly acidic conditions. Its pKa value of 6.0 is well suited for detecting tumor-associated pH changes. Its imaging potential as a spraying agent to detect tumors and determine tumor margin was confirmed using subcutaneous tumor models. Its capability of detecting small-sized ovarian tumors was further demonstrated by IP administration of CypH-11 in a disseminated tumor model.

### Materials and Methods

General information for chemical synthesis. All chemicals and solvents for the synthesis were purchased from Sigma-Aldrich (St. Louis, MO) or Fisher Scientific (Waltham, MA). 4-(N-isopropyl, N-methyl)aminophenol and compound 1, which were used for the synthesis of CypH-11, were synthesized via the reported procedures with necessary modifications. Compound CypH-1 was synthesized as previously reported and Cy7 was purchased from GE Healthcare (Chicago, IL). Both of them were used to compare their imaging capability with CypH-11. Compounds and intermediates were separated and purified with silica gel flash chromatography. ¹H and ¹³C NMR spectra were collected on Bruker Ascend-500 spectrometer and high-resolution mass spectroscopy (HRMS) was collected on a PE Sciex API 100 mass spectrometer.

Synthesis and characterization of CypH-11. To a solution of 4-(N-isopropyl, N-methyl)aminophenol (81 mg, 0.491 mmol) in anhydrous N,N-dimethylformamide (DMF, 5 mL), sodium hydride (NaH, 22 mg, 60% in oil, 0.55 mmol) was added. The reaction stirred for 15 minutes to form the sodium phenoxide salt. Compound 1 (150 mg, 0.225 mmol) was then added and the mixture stirred at room temperature overnight. Upon completion, the DMF solvent was removed under vacuum. The residue was purified using silica gel chromatography with an increasing gradient of methanol in dichloromethane (0-6%). The desired CypH-11 compound was acquired as a green solid (0.051 mg, 28.5%). ¹H NMR (500 MHz, CDCl₃) δ 7.99 (d, J=14.1 Hz, 2H), 7.33-7.24 (m, 6H), 7.20-7.13 (m, 2H), 6.95 (d, J=9.1 Hz, 2H), 6.82 (d, J=9.1 Hz, 2H), 6.21 (d, J=14.1 Hz, 2H), 4.96-4.87 (m, 2H), 3.94-3.86 (m, 1H), 2.76 (t, J=6.0 Hz, 4H), 2.63 (s, 3H), 2.10-2.00 (m, 2H), 1.66 (d, J=7.0 Hz, 12H), 1.35 (s, 12H), 1.07 (d, J=6.6 Hz, 6H). ¹³C NMR (126 MHz, CDCl₃) δ 171.81, 165.23, 152.98, 146.18, 142.71, 141.85, 141.05, 128.22, 124.54, 123.07, 122.26, 116.84, 114.98, 112.76, 100.48, 50.97, 48.94, 48.75, 30.63, 28.19, 24.77, 21.22, 19.72, 18.83. ESI-HRMS: for [C₄₆H₅₈N₃O]⁺: expected m/z=668.4580 [M]⁺; found m/z=668.4557 [M]⁺; 3.4 ppm error.

Spectroscopic analysis. Stock solutions (1 mM in DMSO) of CypH-11, CypH-1, and Cy7 were stored at a −30° C. freezer and used for the following experiments. For the pKa measurement, the respective compound was diluted in 20 mM phosphate-buffered solution (PBS, pH 2.0-11.0) to a final concentration of 2 μM. The fluorescence intensity (λₑₓ=725 nm and λ_{cm}=785 nm) was measured on a plate reader (Tecan Infinite M1000 Pro), and the fluorescence images were recorded with a fluorescence imaging system (Bruker In-vivo F Pro). For the quantum yield measurement, indocyanine green (ICG, Φ=1.2% in water) was used as the standard compound. The respective compound (0.4, 0.8, 1.2, 1.6, and 2.0 μM) in PBS solutions (pH=4.0 and pH=7.4) was measured using a Cary 60 UV-Vis spectrophotometer and Cary Eclipse fluorescence spectrophotometer (Agilent). The slope of fluorescence vs absorbance was compared with that of ICG to calculate the relative quantum yield.

Cell lines and culture. The ovarian cancer OVASAHO cell line was purchased from JCRB cell bank (Osaka, Japan), OVSAHO/RFP-Luc cell was transduced with FLus-F2A-RFP-IRES-Puro Lentivirus (Biosettia, San Diego, CA) and selected with puromycin, and SKOV3/GFP-Luc cell was purchased from Cell Biolabs (San Diego, CA). Both OVASAHO and OVASAHO/RFP-Luc cells were maintained in RPMI1640 medium supplemented with 10% fetal bovine serum (FBS) and 1% penicillin/streptomycin at 37° C. under 5% CO₂, and SKOV3/GFP-Luc cells were maintained in McCoy's 5A medium supplemented with 10% fetal bovine serum (FBS) and 1% penicillin/streptomycin at 37° C. under 5% CO₂.

In vitro fluorescence microscopy. OVASAHO cells were used to compare the cellular performance and cellular distribution of CypH-11, CypH-1, and Cy7. OVASAHO cells (1.0×10⁴) were seeded on a 96-well black plate (Corning, NY) and incubated in the supplemented medium for 24 hrs. Compounds (2 μM) were added and the cells were incubated for 1 hr. Before PBS washing, the cellular fluorescence images were captured with a fluorescence microscope (Cy7 filter, excitation: 690-730 nm and emission: 770-850 nm). After PBS washing (3×), the cellular images were captured again. For the co-localization experiment, OVASAHO cells (5.0×10³) were incubated on a 96 well plate (ibiTreat, 180 μm coverslip, ibidi) with a clear and flat bottom. After treatment with CypH-11 (2 μM) or CypH-1 (2 μM) for 1 hr, the cells were washed with medium (3×). The cells were stained with Mitoview Green (Biotium, Parkway Fremont, CA) for 15 min or Lysoview 488 (Biotium) for 30 min. After washing with medium (3×), cells were further stained with DAPI (Invitrogen) for 10 min. The fluorescence images were captured using a fluorescence microscope (EVOS) after replacing the medium with a fresh cell culture medium. CypH-11 and CypH-1 images were obtained with a Cy7 filter, DAPI images with a DAPI filter (excitation: 352-402 nm and emission: 417-477 nm), Mitoview Green and Lysoview 488 images with a GFP filter (excitation: 457-487 nm and emission: 502-538 nm).

Cell viability. The cell viability of CypH-11 and CypH-1 were evaluated using the cell counting kit-8 (CCK-8) from Dojindo (Rockville, MD). OVASAHO cells (5.0×10³) were seeded on a 96-well plate and cultured for 24 hrs. Then the cells were treated with CypH-11 (2 μM) and CypH-1 (2 μM) for 1 hr. After replacing the medium with fresh cell culture medium, the cells were incubated for another 72 hrs. The cell viability was examined by treating them with CCK-8 solution for 3 hrs and reading the absorbance at 450 nm using a plate reader.

Tumor models of subcutaneous implants and peritoneal implants. All animal procedures were carried out in compliance with the approved animal protocols and guidelines of the Institutional Animal Care and Use Committee at Weil Cornell Medical College. Mice (female, SCID Hairless Outbred Mouse) were purchased from Charles River (Wilmington, MA). To establish the subcutaneous implants, a suspension of OVASAHO/RFP-Luc cells (5.0×10⁶) or SKOV3/GFP-Luc cells (5.0×10⁶) in 100 μL PBS was inoculated into each flank of the female nude mice on both sides. After two weeks, the tumor implants grew to around 5 mm in size and were used for the spray experiment. To establish the peritoneal implants, SKOV3/GFP-Luc cells (5.0×10⁶) suspended in 200 PBS were injected into the abdominal cavities of the female nude mice. After two weeks, the tumor growth was examined with an in vivo bioluminescence imaging system followed by peritoneal injection of D-luciferin potassium solution (200 μL, 15 mg/mL) for 10 min. Generally, mice bearing multiple disseminated peritoneal implants of 5 mm size in diameter were used for the experiment.

In vivo fluorescence imaging of subcutaneous tumors. Two subcutaneous tumor models (OVASAHO/RFP-Luc and SKOV3/GFP-Luc) were used to compare the imaging capability of CypH-11, CypH-1, and Cy7. Mice bearing the subcutaneous implants were anesthetized in an induction chamber using 2% isoflurane, and anesthesia was maintained with 1.5-2.0% isoflurane via a nose cone. Sterile surgical tools were used to remove skin around the tumor. The mice images were captured using an IVIS SpectrumCT System from PerkinElmer (Waltham, MA). OVASAHO/RFP-Luc and SKOV3/GFP-Luc tumors were captured under an RFP channel (Excitation: 520-550 nm and Emission: 570-590 nm) and a GFP channel (Excitation: 450-480 nm and Emission: 510 530 nm), respectively. A Cy7 channel (Excitation: 730-760 nm and Emission: 790-810 nm) was applied to evaluate the fluorescence generated by CypH-11, CypH-1, and Cy7. After skin removal, the tumor area and the background fluorescence under the Cy7 channel were measured first. Solutions of CypH-11, CypH-1, and Cy7 (2 μM each) were sprayed on the surgical area, and the fluorescence of the Cy7 channel was measured continuously at each time point (1, 3, 5, 7, 10, and 15 min). Ten, five, and three OVASAHO tumors were used to evaluate CypH-11, CypH-1, and Cy7, respectively. Six SKOV3 tumors were used to evaluate CypH-11. To evaluate the tumor-to-normal tissue ratio, the whole tumor regions and the adjacent open-skin areas were drawn, and their fluorescence intensity was acquired by the IVIS software. Tumor-to-normal tissue ratio value was calculated as the average fluorescence intensity of the tumor divided by that of the normal area. Also, tumor-bearing mice were euthanized by carbon dioxide inhalation or a high dose of isoflurane (5%). The subcutaneous tumors and the adjacent muscles were then extracted, and CypH-11 (2 μM) was sprayed onto them. Images were subsequently captured under GFP/RFP/Cy7 channels.

In vivo fluorescence imaging of disseminated peritoneal tumors. To further evaluate the imaging capability of CypH-11 highlighting the disseminated tiny metastasis in the peritoneal cavity, SKOV3/GFP-Luc tumors were implanted and allowed to grow and disseminate in the mouse peritoneal cavity, which is similar to those of ovarian cancer patients. Tumor-bearing mice were injected intraperitoneally with the CypH-11 solution in PBS (200 μL, 2 μM). After 1 hr, the mice were anesthetized in an induction chamber using 2% isoflurane, and anesthesia was maintained via a nose cone with 1.5% to 2% isoflurane. Sterile surgical tools were used to open the abdomen cavities. The fluorescence images were captured for the whole cavity under both the GFP and Cy7 channels. After imaging, mice were euthanized with a high dose of isoflurane (5%), and the disseminated tumors and the main organs of interest (i.e., heart, liver, lung, kidneys, spleen, stomach, and intestine) were collected. The collected organs were placed on a glass plate and imaged under both the GFP and Cy7 channels. Regions of interest (ROI, n=6, and average area=0.28±0.1 cm²) within the tumor nodules and in the adjacent normal areas in the peritoneal cavity were drawn and calculated for tumor-to-normal tissue ratio.

Histology. To gain knowledge about the CypH-11 distribution in tumors after administration via spray and i.p. injection, the tumors were excised and analyzed. The tumors were firstly embedded into a mold with optimal cutting temperature (OCT) compound (Tissue-Tek, Sakura Finetek, Torrance, CA) on dry ice for 20 min. The frozen tissue was sectioned into the desired thickness (14 μm) using a cryotome. The slides were stored at −80° C. until further use. The slides were imaged first with a fluorescence microscope (EVOS, Thermofisher Scientific, Waltham, MA) and were subsequently stained with hematoxylin and eosin Y solution (H&E) to assess their histologic alterations under a light microscope.

Statistical analysis. Cytotoxicity, fluorescent ratio, and histological analyses were applied to the unpaired t-test. All p-values are two-tailed, and p-values<0.05 were considered significant. Plotted values are represented as mean±standard deviation. Statistical analyses were performed using the GraphPad Prism (GraphPad Software Inc, San Diego, CA).

Although the present disclosure has been described with respect to one or more particular embodiments and/or examples, it will be understood that other embodiments and/or examples of the present disclosure may be made without departing from the scope of the present disclosure.

1. A compound having the following structure: 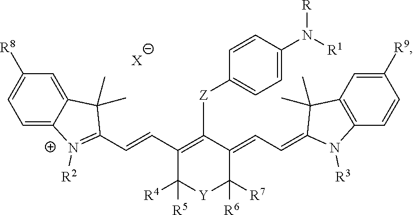
wherein
X is an anion;
Y is NH, NR¹⁰, or CR¹¹R¹²;
Z is a heteroatom chosen from O, S, or Se;
R and R¹ are independently chosen from methyl, ethyl, propyl, isopropyl, butyl, isobutyl, n-butyl, t-butyl, and combinations thereof;
R² and R³ are independently chosen from meth methyl, ethyl, propyl, isopropyl, butyl, isobutyl, n-butyl, t-butyl, and combinations thereof;
R⁴, R⁵, R⁶, R⁷, R⁸, R⁹, R¹⁰, R¹¹, and R¹² are independently chosen from hydrogen, alkyl groups, and combinations thereof,
with the proviso the compound does not have the following structure:

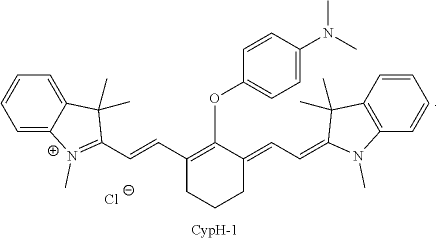 [SMILES: CN(C)c1ccc(OC2=C(/C=C/C3=[N+](C)c4ccccc4C3(C)C)CCC/C2=C\C=C2\N(C)c3ccccc3C2(C)C)cc1 ; [Cl-]]

2. A compound of claim 1, wherein the compound has the following structure:

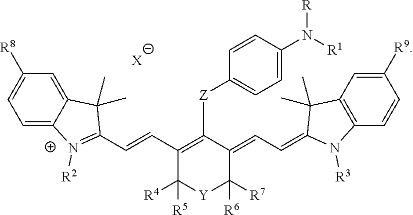

3. A compound of claim 2, wherein the compound has the following structure

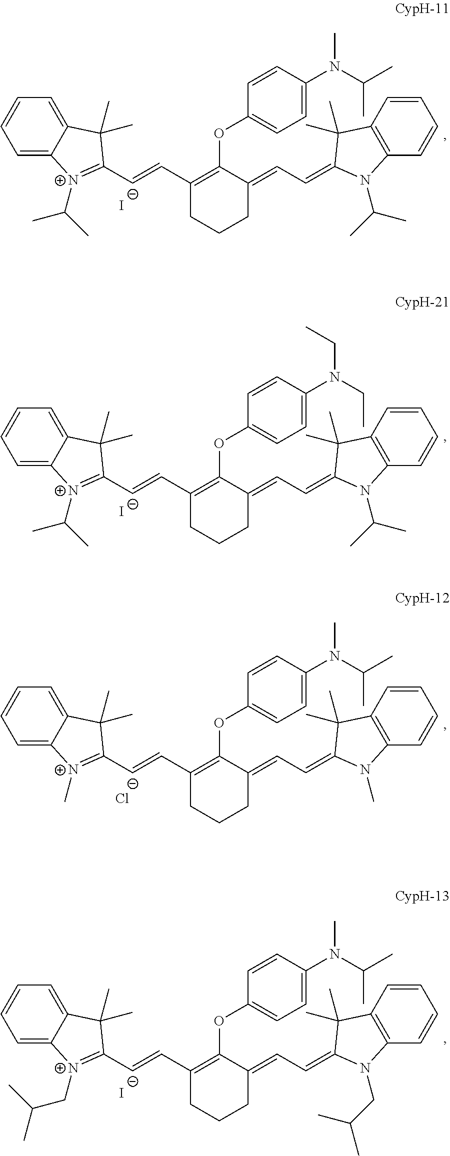

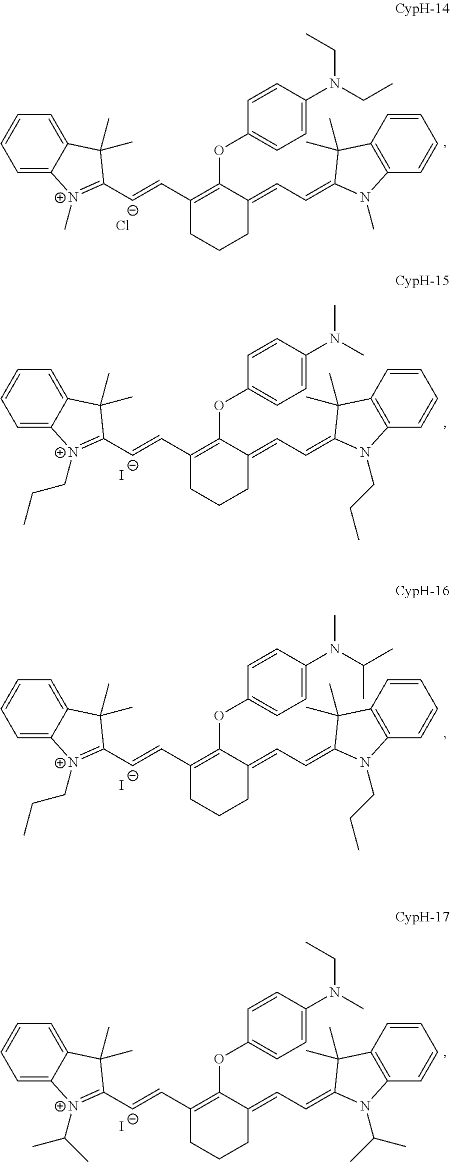

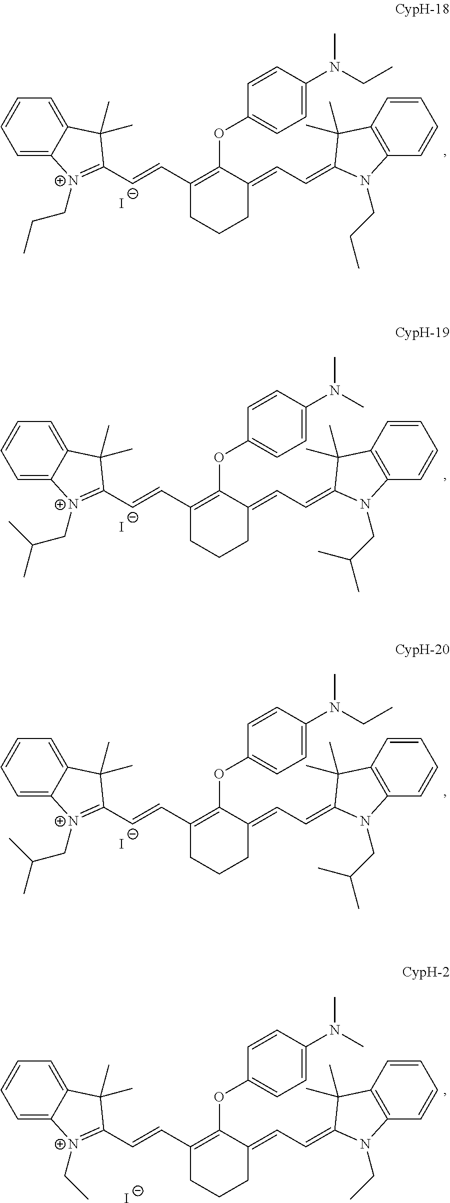

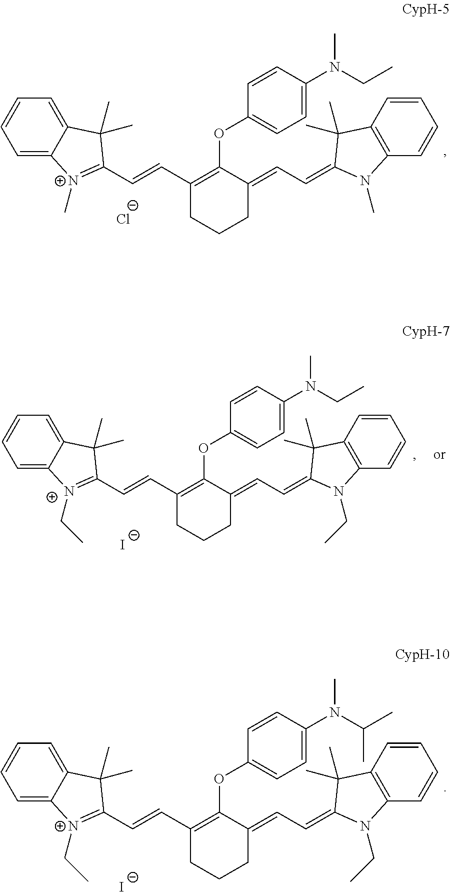 [SMILES: CCN(C)c1ccc(OC2=C(/C=C/C3=[N+](C)c4ccccc4C3(C)C)CCC/C2=C\C=C2\N(C)c3ccccc3C2(C)C)cc1 ; [Cl-] ; CCN(C)c1ccc(OC2=C(/C=C/C3=[N+](CC)c4ccccc4C3(C)C)CCC/C2=C\C=C2\N(CC)c3ccccc3C2(C)C)cc1 ; [I-] ; CCN1/C(=C/C=C2\CCCC(/C=C/C3=[N+](CC)c4ccccc4C3(C)C)=C2Oc2ccc(N(C)C(C)C)cc2)C(C)(C)c2ccccc21 ; [I-]]

4. A compound of claim 3, wherein the compound has the following structure:

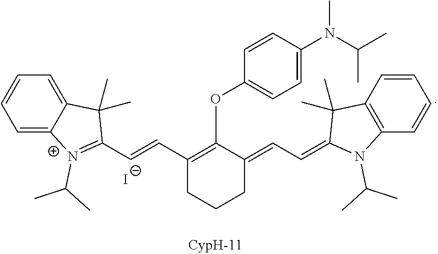 [SMILES: CC(C)N(C)c1ccc(OC2=C(/C=C/C3=[N+](C(C)C)c4ccccc4C3(C)C)CCC/C2=C\C=C2\N(C(C)C)c3ccccc3C2(C)C)cc1 ; [I-]]

5. A composition comprising a compound of claim 1 and a pharmaceutically acceptable carrier.

6. The composition of claim 5, wherein the concentration of the compound is 0.5 to 10 μM.

7. The composition of claim 5, wherein the composition is a sprayable composition or oral rinse.

8. A method of determining the presence and/or location of a solid tumor in an individual in need of treatment comprising:
administering or applying a compound of claim 1 or a composition comprising the compound to an area of interest on/in the individual;
exposing the area of interest to near infrared electromagnetic radiation; and
imaging and/or visualizing the area of interest,
wherein the presence and/or location of the solid tumor is determined.

9. The method of claim 8, wherein a signal is generated from the exposing and the signal is detected in less than 5 minutes.

10. The method of claim 9, wherein the signal is detected in less than 1 minute.

11. The method of claim 8, wherein the administering/applying is a topical administration/application.

12. The method of claim 11, wherein the topical administration/application is spraying or an oral rinsing.

13. The method of claim 8, wherein the solid tumor is chosen from ovarian tumors, skin cancer, pancreatic cancer, genitourinary cancer, colon tumors, bladder tumors, brain tumors, esophagus tumors, cervical tumors, oral tumors, and combinations thereof.

14. The method of claim 8, wherein the administration/application to the solid tumor results in the protonation of the compound.

15. A method of imaging a solid tumor comprising:
applying or administering a compound of claim 1 to the solid tumor;
exposing an area of interest to near-infrared electromagnetic radiation; and
obtaining an image of the solid tumor.

16. The method of claim 15, wherein after application or administration of the compound, wherein a signal is generated from the exposing and the signal is detected in less than 5 minutes.

17. The method of claim 16, wherein the signal is detected in less than 1 minute.

18. The method of claim 17, wherein the administering/applying is a topical administration/application.

19. The method of claim 16, wherein the topical administration/application is spraying or an oral rinsing.

20. The method of claim 16, wherein the solid tumor is chosen from ovarian tumors, skin cancer, pancreatic cancer, genitourinary cancer, brain tumors, colon tumors, bladder tumors, esophagus tumors, cervical tumors, oral tumors, and combinations thereof.

21. A method for determining margins of a solid tumor, comprising:
applying or administering a compound of claim 1 to the solid tumor;
exposing an area of interest to near-infrared electromagnetic radiation;
imaging and/or visualizing the area of interest; and
determining the margin of the solid tumor,
wherein the margins of the solid tumor against non-cancerous tissue is determined.

22. The method of claim 21, wherein the applying or administering is spraying or an oral rinsing.

23. The method of claim 21, wherein a signal is generated from the exposing and the signal is detected in less than 5 minutes.

24. The method of claim 23, wherein the signal is detected in less than 1 minute.

25. The method of claim 21, wherein the solid tumor is chosen from ovarian tumors, skin cancer, pancreatic cancer, genitourinary cancer, brain tumors, colon tumors, bladder tumors, esophagus tumors, cervical tumors, oral tumors, and combinations thereof.

26. A kit comprising a compound of claim 1.
